(19) United States
(12) Patent Application Publication
(10) Pub. No.: US 2014/0194800 A1
(43) Pub. Date: Jul. 10, 2014

(54) GROOVED GRIP

(71) Applicant: Travis GREEN, Fort Belvoir, VA (US)

(72) Inventor: Travis GREEN, Fort Belvoir, VA (US)

(21) Appl. No.: 14/060,368

(22) Filed: Oct. 22, 2013

Related U.S. Application Data
(60) Provisional application No. 61/750,485, filed on Jan. 9, 2013.

(51) Int. Cl. B25G 1/10; A61F 5/01; G06F 3/0354

(52) U.S. Cl. CPC B25G 1/102; G06F 3/03543; A61F 5/013
USPC 602/21; 16/430

(57) ABSTRACT

Disclosed is a grooved grip having a body configured to be grasped by a hand comprising a channel configured to relieve pressure on a median nerve on a palmar surface on a hand.

### CROSS-REFERENCE TO RELATED APPLICATIONS

This application claims the benefit of U.S. Provisional Application No. 61/750,485, filed Jan. 9, 2013, entitled “GROOVED CANE,” which is hereby incorporated by reference in its entirety.

### BRIEF DESCRIPTION OF THE SEVERAL VIEWS OF THE DRAWINGS

FIG. A1 shows a perspective view of a left-handed grooved grip, according to an embodiment.

FIG. A2 shows a back elevational view thereof.

FIG. A3 shows a front elevational view thereof.

FIG. A4 shows a left elevational view thereof.

FIG. A5 shows a right elevational view thereof.

FIG. A6 shows a top elevational view thereof.

FIG. A7 shows a bottom elevational view thereof.

FIG. A8 shows a bottom perspective view thereof.

FIG. B1 shows a perspective view of a grooved fixed pivot ambidextrous grip, according to an embodiment.

FIG. B2 shows a front elevational view thereof.

FIG. B3 shows a back elevational view thereof.

FIG. B4 shows a top elevational view thereof.

FIG. B5 shows a cross-sectional view taken along section B-B of FIG. B4.

FIG. B6 shows a bottom elevational view thereof.

FIG. B7 shows a right elevational view thereof.

FIG. B8 shows a left elevational view thereof.

FIG. B9 shows a perspective view of the grooved fixed pivot ambidextrous grip, according to the embodiment, connected to a shaft of a cane.

FIG. C1 shows a perspective view of a grooved pivoting ambidextrous grip, according to an embodiment.

FIG. C2 shows a front elevational view thereof.

FIG. C3 shows a back elevational view thereof.

FIG. C4 shows a top elevational view thereof.

FIG. C5 shows a cross-sectional view taken along section C-C of FIG. C4.

FIG. C6 shows a bottom elevational view thereof.

FIG. C7 shows a right elevational view thereof.

FIG. C8 shows a left elevational view thereof.

FIG. C9 shows a perspective view of the grooved pivoting ambidextrous grip, according to the embodiment, connected to a shaft of a cane.

FIG. D1 shows a perspective view of a grooved glove, according to an embodiment.

FIG. D2 shows a front view thereof.

FIG. D3 shows a back view thereof.

FIG. D4 shows a top view thereof.

FIG. D5 shows a cross-sectional view taken along section D-D of FIG. D4.

FIG. D6 shows a bottom view thereof.

FIG. D7 shows a right side view thereof.

FIG. D8 shows a left side view thereof.

FIG. E1 shows a perspective view of a grooved pivoting steering wheel spinner grip, according to an embodiment.

FIG. E2 shows a front elevational view thereof.

FIG. E3 shows a back elevational view thereof.

FIG. E4 shows a top elevational view thereof.

FIG. E5 shows a bottom elevational view thereof.

FIG. E6 shows a right elevational view thereof.

FIG. E7 shows a left elevational view thereof.

FIG. E8 shows a perspective view of a grooved pivoting steering wheel spinner grip, according to the embodiment connected to a steering wheel.

FIG. F1 shows a perspective view of a left-handed grooved steering wheel spinner grip, according to an embodiment.

FIG. F2 shows a back elevational view thereof.

FIG. F3 shows a front elevational view thereof.

FIG. F4 shows a left elevational view thereof.

FIG. F5 shows a right elevational view thereof.

FIG. F6 shows a top elevational view thereof.

FIG. F7 shows a bottom devotional view thereof.

FIG. F8 shows a bottom perspective view thereof.

FIG. F9 shows a perspective view of a left-handed grooved steering wheel spinner grip, according to the embodiment, connected to a steering wheel.

FIG. G1 is a flow chart describing a possible process to manufacture a grooved plastic grip, according to an embodiment.

FIG. G2 is a flow chart describing an alternative possible process to manufacture a grooved plastic grip, according to an embodiment.

FIG. G3 is a flow chart describing a possible process to manufacture a grooved wood grip, according to an embodiment.

FIG. G4 is a flow chart describing a possible process to manufacture a grooved metal grip, according to an embodiment.

FIG. G5 is a flow chart describing an alternative possible process to manufacture a grooved metal grip, according to an embodiment.

FIG. H1 is a flow chart describing a possible process to manufacture a grooved foam pad, according to an embodiment.

FIG. H2 is a flow chart describing a possible process to manufacture a grooved silicone pad, according to an embodiment.

FIG. H3 is a flow chart describing a possible process to manufacture a grip with a grooved pad, according to an embodiment.

FIG. H4 is a flow chart describing an alternative possible process to manufacture grip with a grooved pad, according to an embodiment.

FIG. I1 is a flow chart describing a possible process to manufacture a grooved foam pad glove.

FIG. I2 is a flow chart describing a possible process to manufacture a grooved silicone pad glove.

FIG. J1 shows a perspective view of a left-handed grooved pad according to an embodiment.

FIG. J2 shows a back elevational view thereof.

FIG. J3 shows a front elevational view thereof.

FIG. J4 shows a left elevational view thereof.

FIG. J5 shows a right elevational view thereof.

FIG. J6 shows a top elevational view thereof.

FIG. J7 shows a bottom elevational view thereof.

FIG. J8 shows a bottom perspective view thereof.

FIG. J9 shows a perspective view of a left-handed grooved pad attached to a handle.

FIG. K1 shows a perspective view of a right-handed grooved computer trackball, according to an embodiment.

FIG. K2 shows a back elevational view thereof.

FIG. K3 shows a front elevational view thereof.

FIG. K4 shows a left elevational view thereof.

FIG. K5 shows a right elevational view thereof.

FIG. K6 shows a top elevational view thereof.

FIG. K7 shows a bottom elevational view thereof.

FIG. K8 shows a bottom perspective view thereof.

FIG. L1 shows a perspective view of a right-handed grooved computer mouse, according to an embodiment.

FIG. L2 shows a back elevational view thereof.

FIG. L3 shows a front elevational view thereof.

FIG. L4 shows a left elevational view thereof.

FIG. L5 shows a right elevational view thereof.

FIG. L6 shows a top elevational view thereof.

FIG. L7 shows a bottom elevational view thereof.

FIG. L8 shows a bottom perspective view thereof.

FIG. M1 shows a perspective view of a right-handed grooved splint, according to an embodiment.

FIG. M2 shows an inside view of a right-handed grooved splint, according to an embodiment.

### DETAILED DESCRIPTION OF SEVERAL EMBODIMENTS

In an embodiment, a grooved grip configured to be grasped by a hand may incorporate a channel configured to relieve pressure on a median nerve on a palmar surface on a hand. One embodiment of a grooved grip is illustrated in FIGS. A1-A8. In FIG. A2, a grip configured for a left hand may have a body 102. (Note that in another embodiment, the opposite, a right-handed grip may be formed and used.) In the embodiment, the body 102 may be plastic. However, the body 102 may comprise any other material that can be configured into a grip, such as wood or metal. Along the top surface of body 102 as seen in FIG. A6 may be a channel 101 configured to relieve pressure on a median nerve on a palmar surface on a hand. In FIG. A7, the bottom surface of body 102 may terminate at a mounting hole 110 configured for a shaft of a cane. Alternatively, in the embodiment in FIG. F8, the bottom surface of body 102 may terminate at a mounting pin 210. As seen in FIG. F9, mounting pin 210 may be configured to attach to a steering wheel 520 by inserting mounting pin 210 into steering wheel spinner base 515, allowing body 102 to rotate freely perpendicular to mounting pin 210. In an embodiment, mounting pin 210 may be mounted to the steering wheel 520 in any place on the steering wheel 520 (e.g., centered on the handle under the palm). While the embodiment is illustrated as attaching to a shaft of a cane or a steering wheel spinner base, the embodiment may be attached to any structure configured to receive the embodiment, including a tool (e.g., a drill handle, an impact wrench handle, a hammer handle), exercise/sport equipment (e.g., a hand-cycle handle, a bicycle handle), a crutch handle, and machines (e.g., a lawnmower handle).

An alternative embodiment of a grooved grip is illustrated in FIGS. B1-B9. In FIG. B2, a grooved fixed pivot ambidextrous grip may have a body 202 to be grasped by either a left hand or right hand. (Note that, in other embodiments, a left handed or right handed fixed pivot grip may be formed or used.) In FIG. B3, the body 202 may have a recess 205 to be grasped by a finger of a hand. In FIG. B4, a channel 201 configured to relieve pressure on a median nerve on a palmar surface on a hand may be along the top surface of body 202. In FIG. B5, the bottom surface of body 202 may terminate at a mounting pin 210. In FIG. B9, mounting pin 210 may be attached to a shaft of a cane 215 or the like. While the embodiment is illustrated as attaching to a shaft of a cane 215, the embodiment may be attached to any structure configured to receive the embodiment, including a tool (e.g., a drill handle, an impact wrench handle, a hammer handle), exercise/sport equipment (e.g., a hand-cycle handle, a bicycle handle), a crutch handle, and machines (e.g., a lawnmower handle).

An alternative embodiment of a grooved grip is illustrated in FIGS. C1-C9. In FIG. C2, a grooved pivoting ambidextrous grip may have a head 302 to be grasped by either a left hand or right hand. (Note that, in other embodiments, a left handed or right handed pivoting grip may be used.) A channel 301 configured to relieve pressure on a median nerve on a palm surface of a hand may be along the top surface of head 302. The head 302 may be connected to a body 303 at a pivot joint 304. In FIG. C5, the pivot joint 304 may be a socket and pin joint that allows movement of the head 302 perpendicular to the body 303. However, pivot joint 304 may comprise any other type of pivot joint that allows one axis of motion of the head 302 with respect to the body 303. In the embodiment in FIG. C8, the bottom surface of a body 303 may terminate at a mounting pin 310. In the embodiment in FIG. C9, the mounting pin 310 may be attached to a shaft of a cane 315. Alternatively, in FIG. E8, the mounting pin 510 may be configured to attach to a steering wheel 520 at a steering wheel column base 515. While the embodiment is illustrated as attaching to a shaft of a cane 315 or a steering wheel column base 515, the embodiment may be attached to any structure configured to receive the embodiment, including a tool (e.g., a drill handle, an impact wrench handle, a hammer handle), exercise/sport equipment (e.g., a hand-cycle handle a bicycle handle), a crutch handle, and machines (e.g., a lawnmower handle).

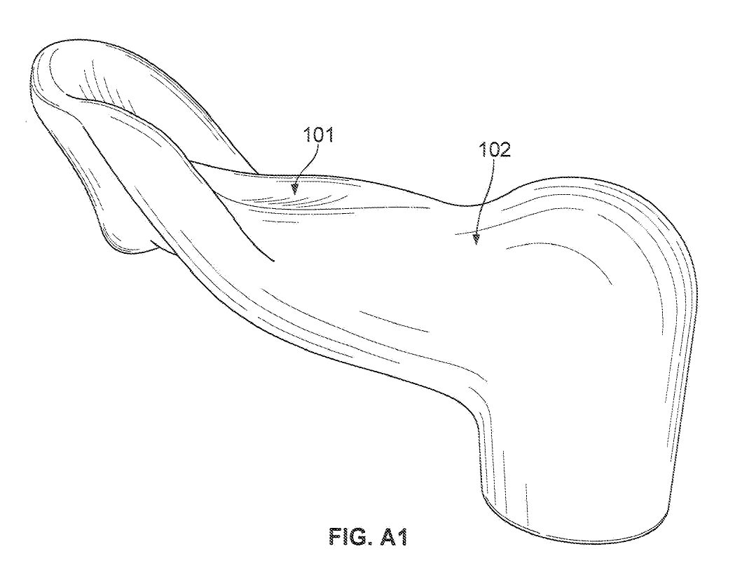

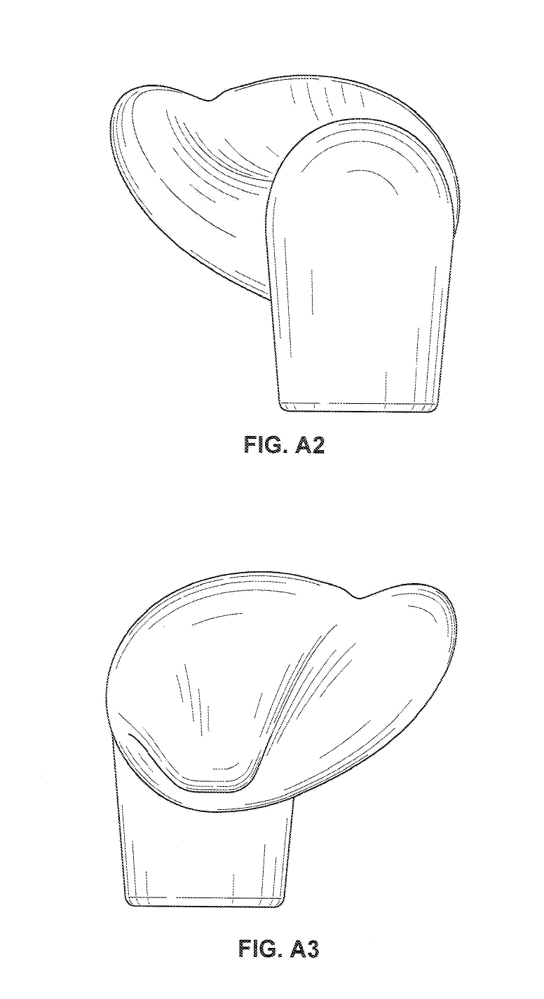

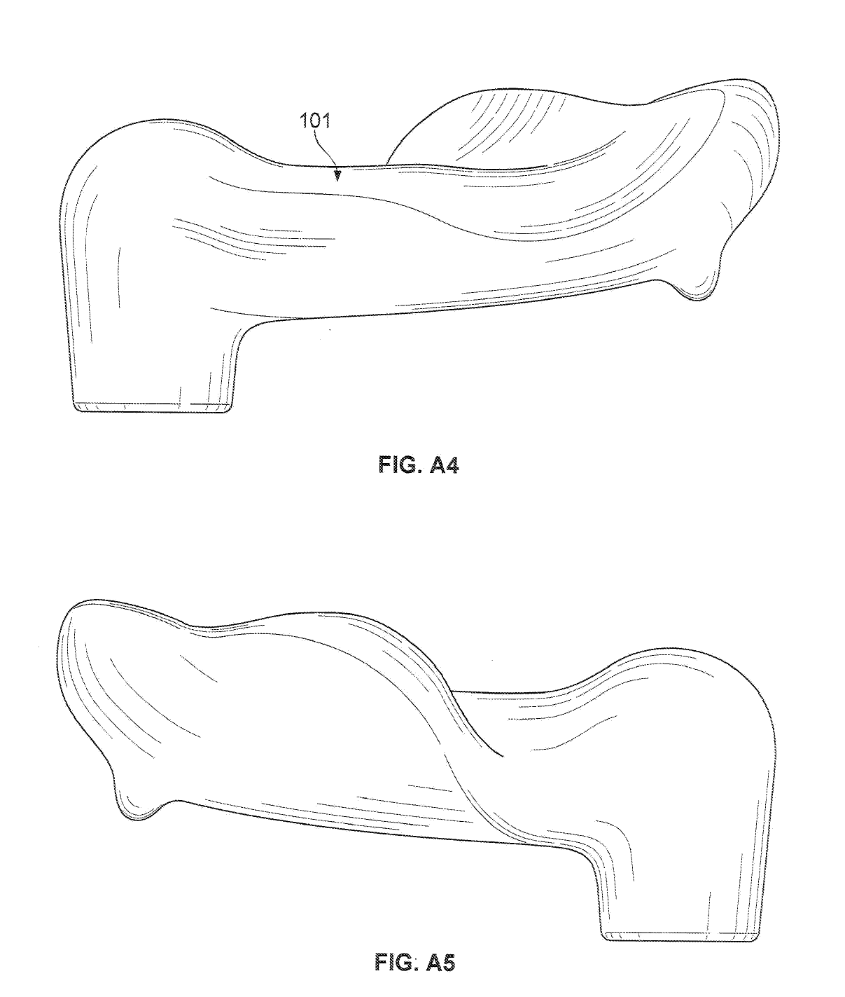

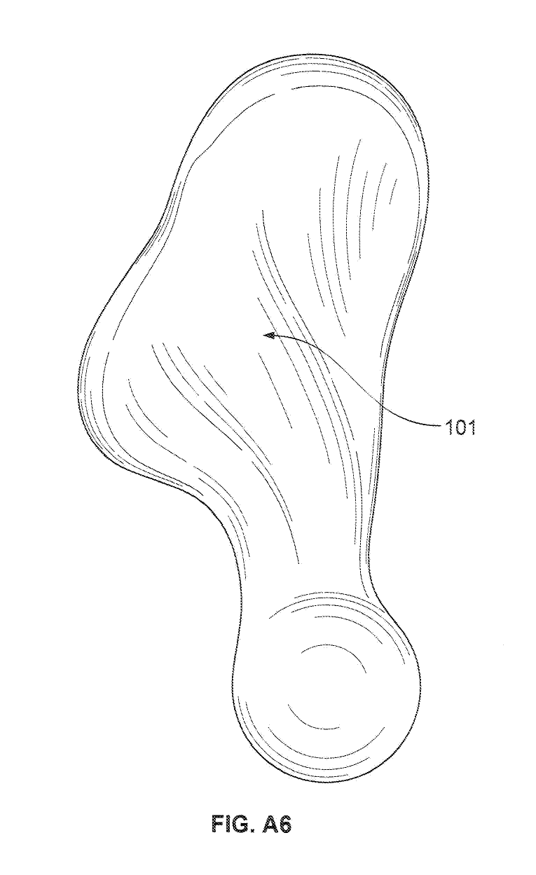

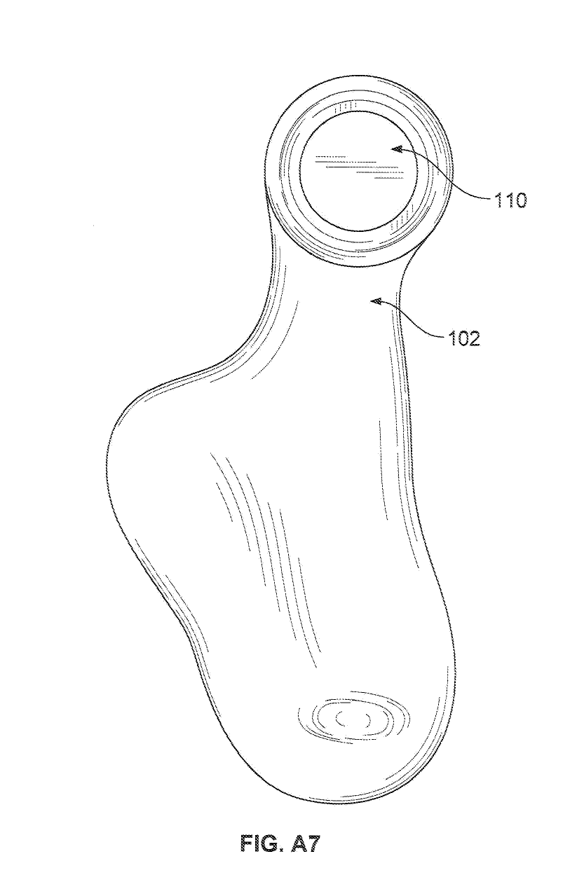

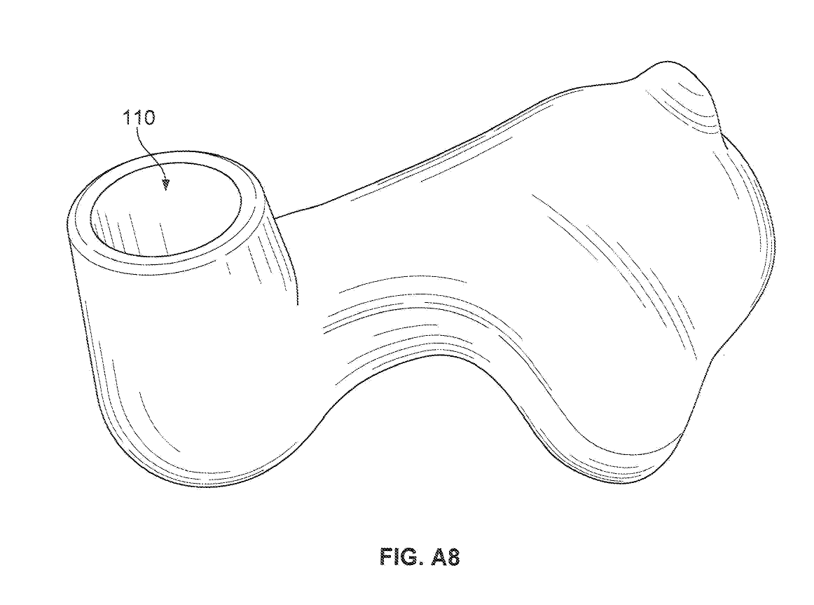

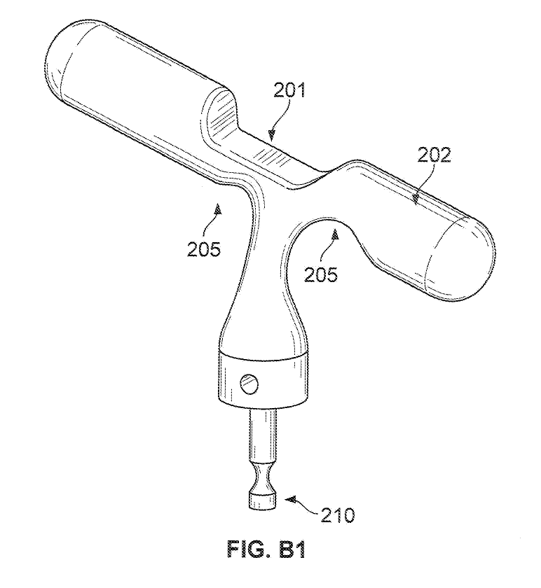

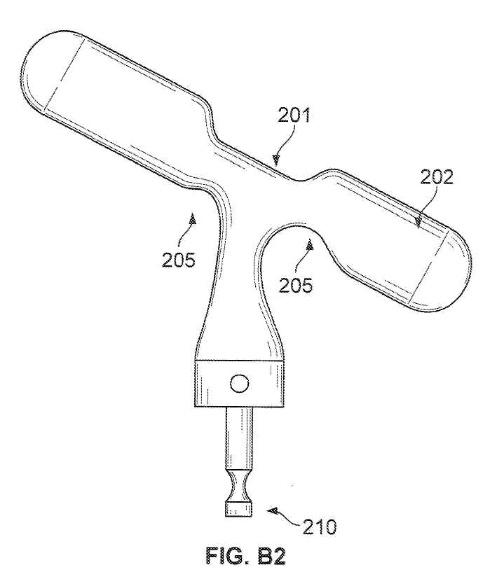

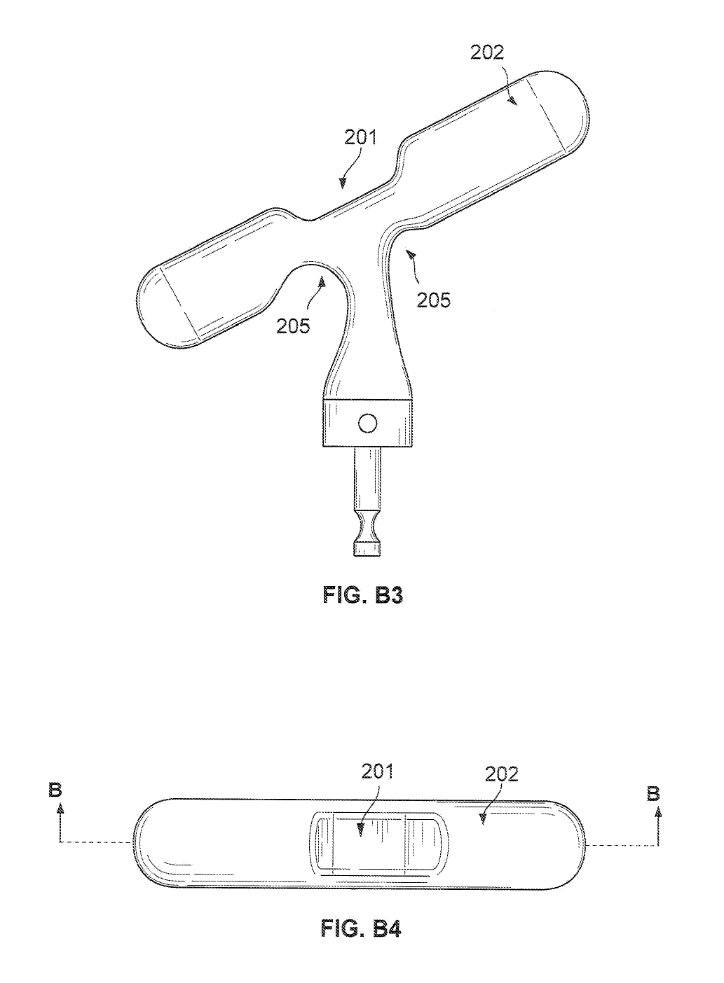

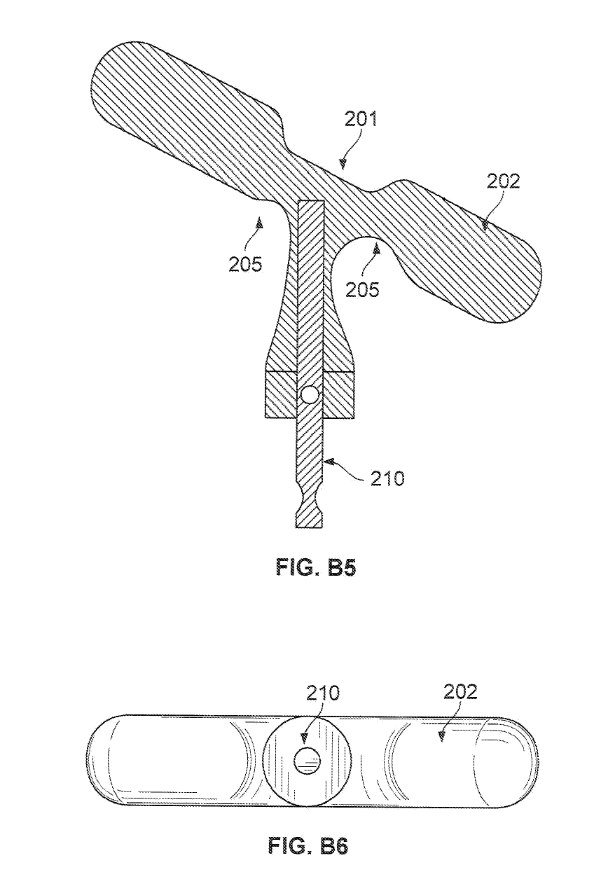

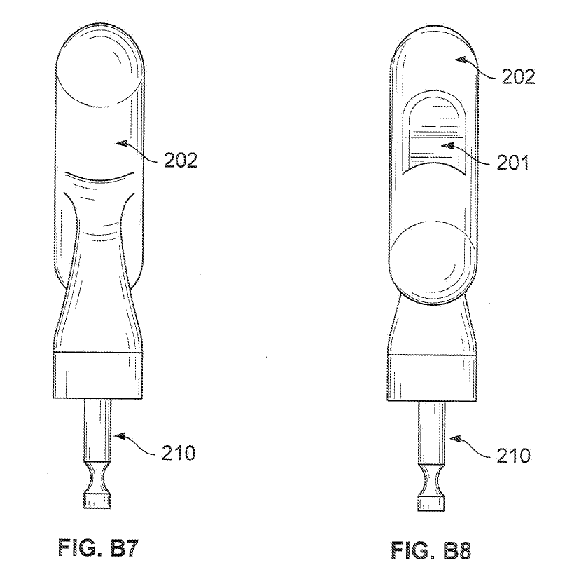

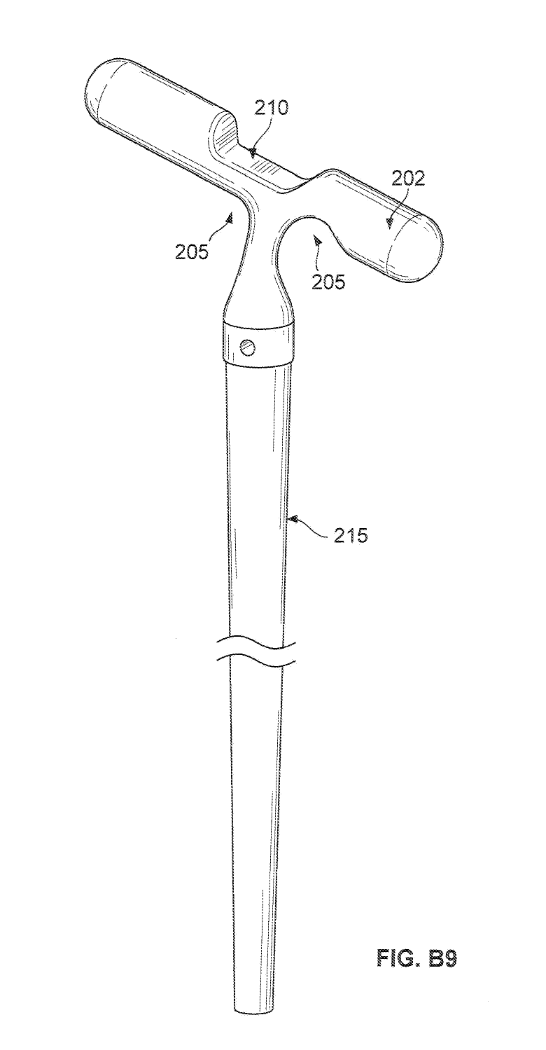

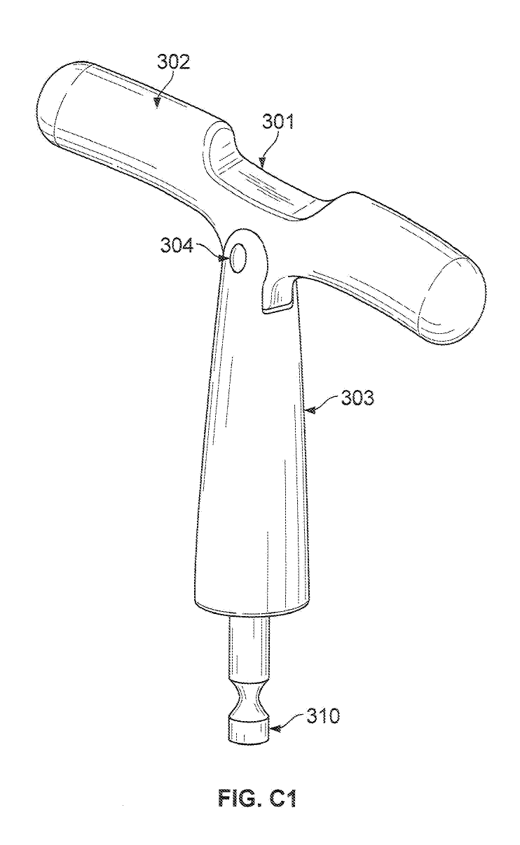

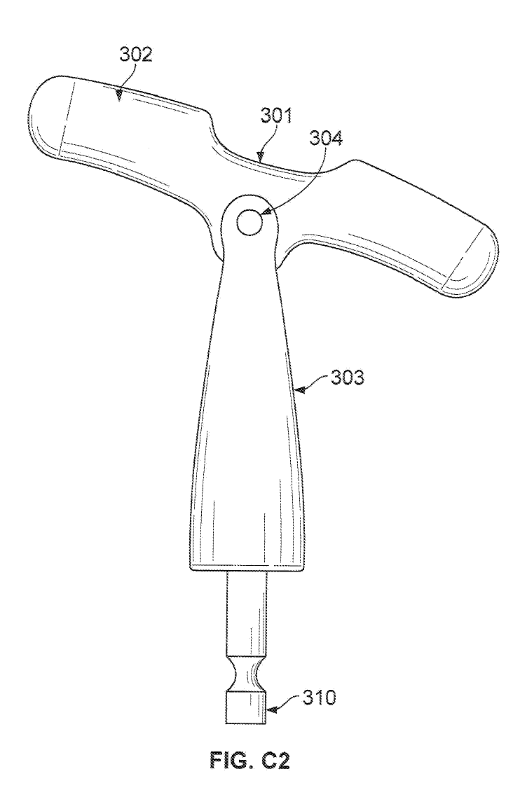

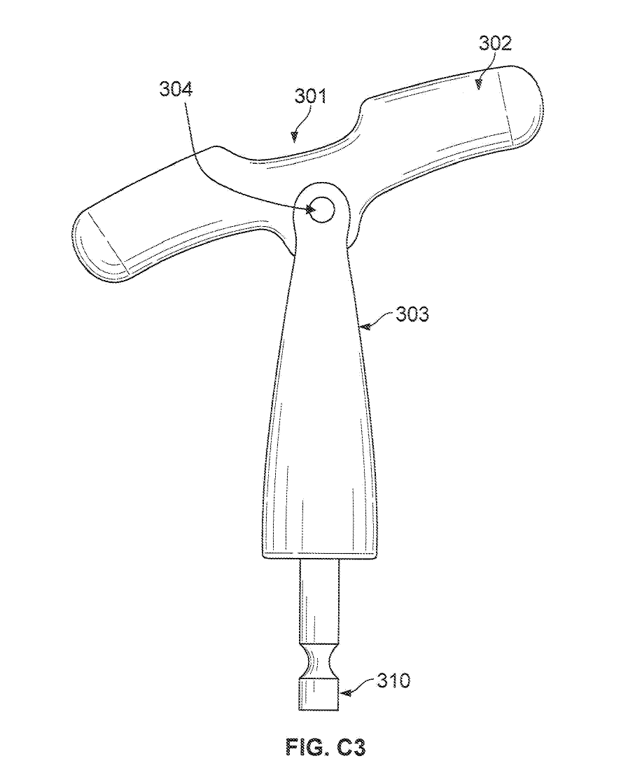

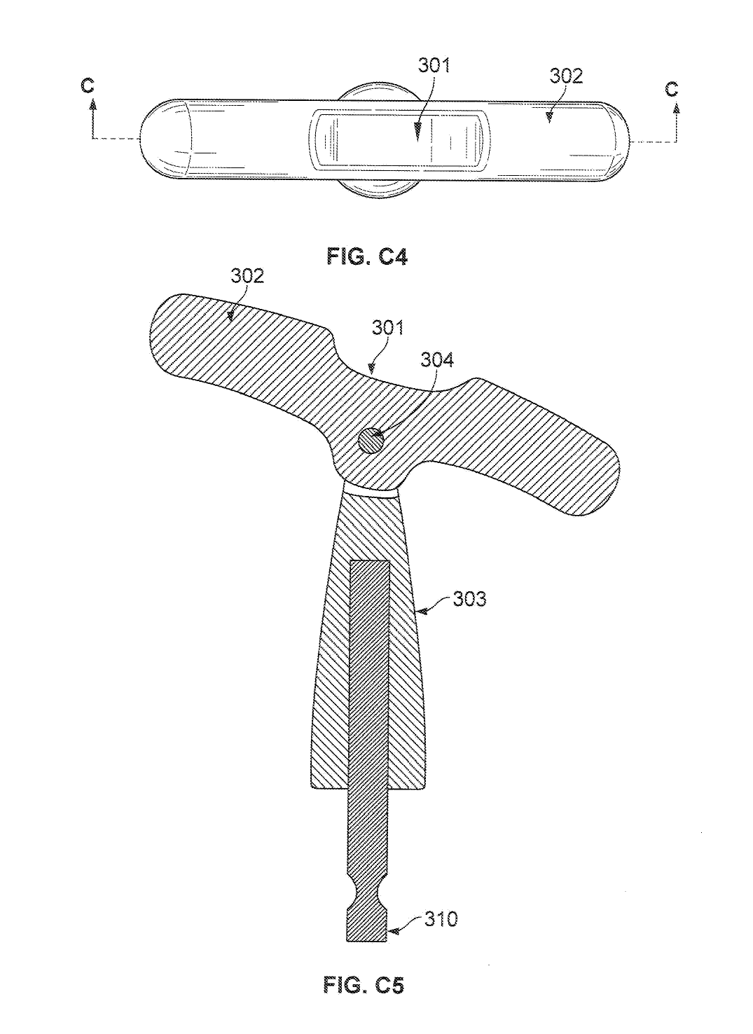

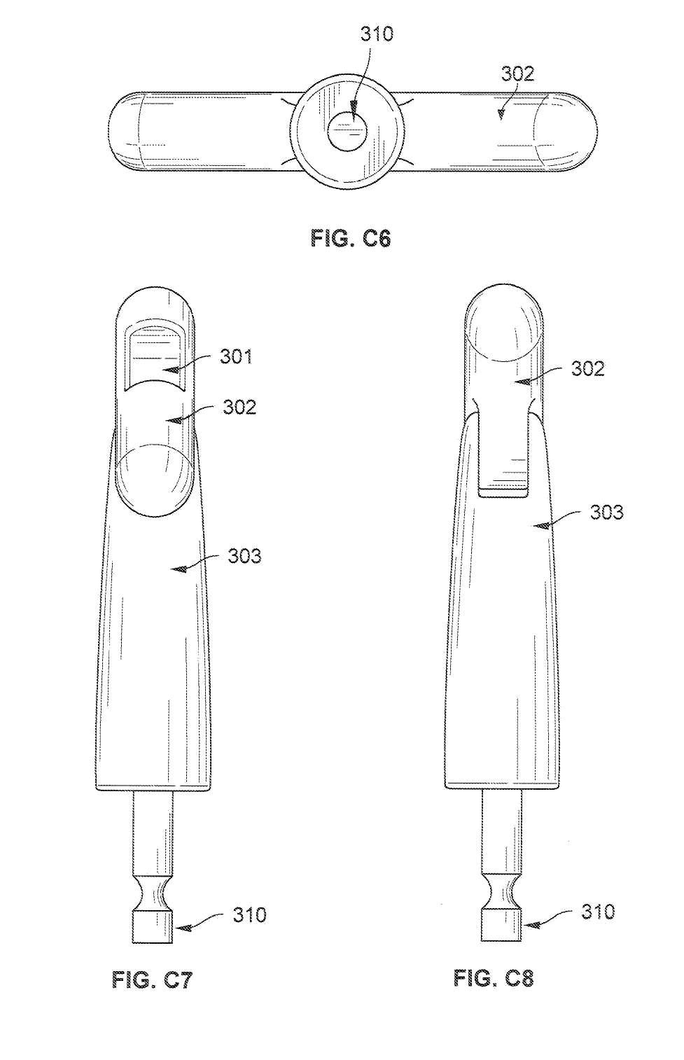

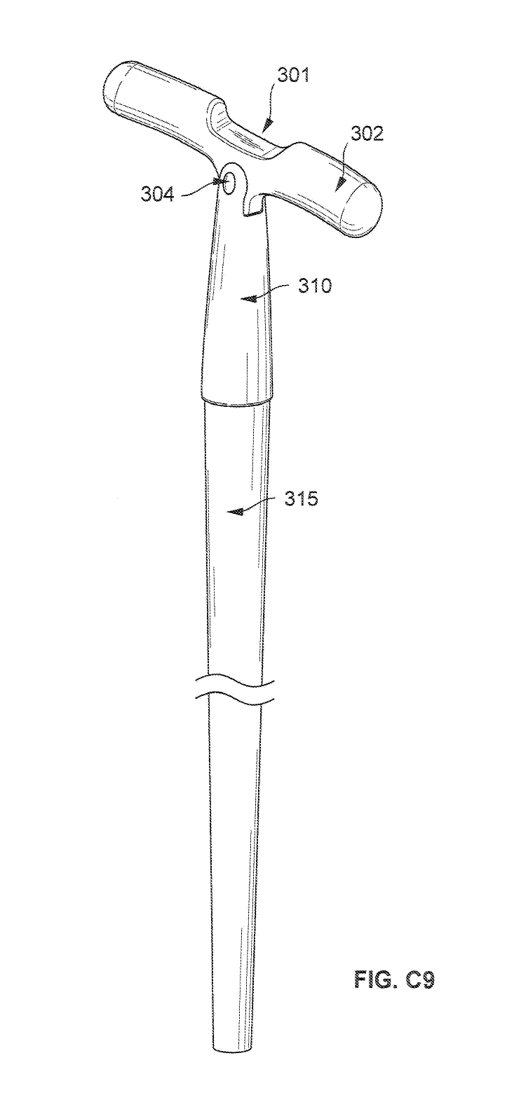

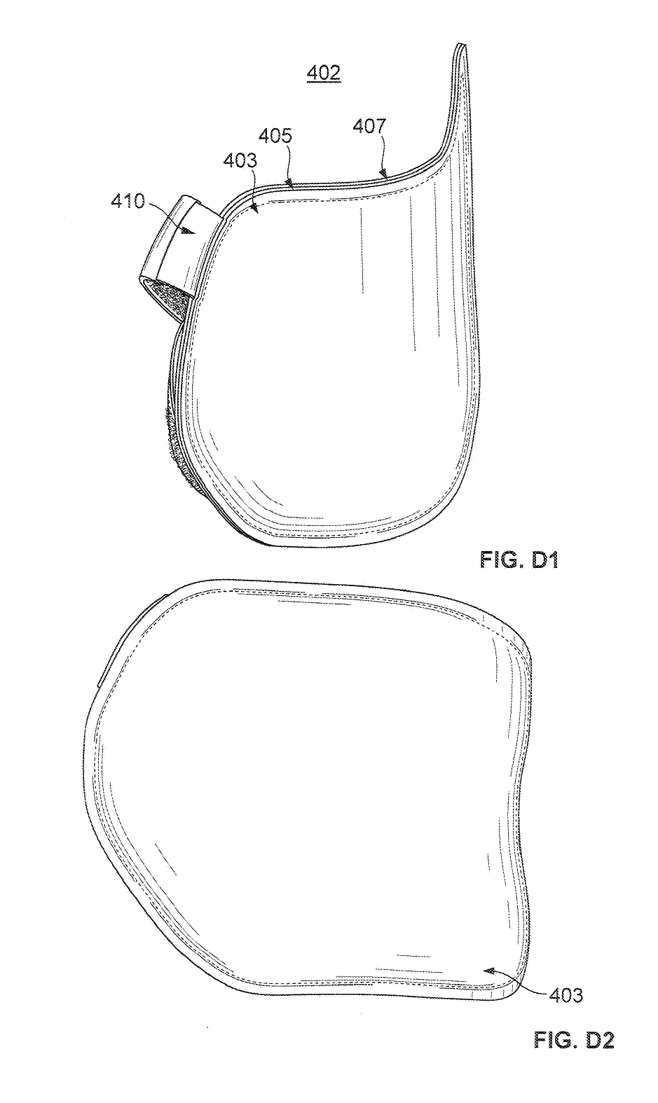

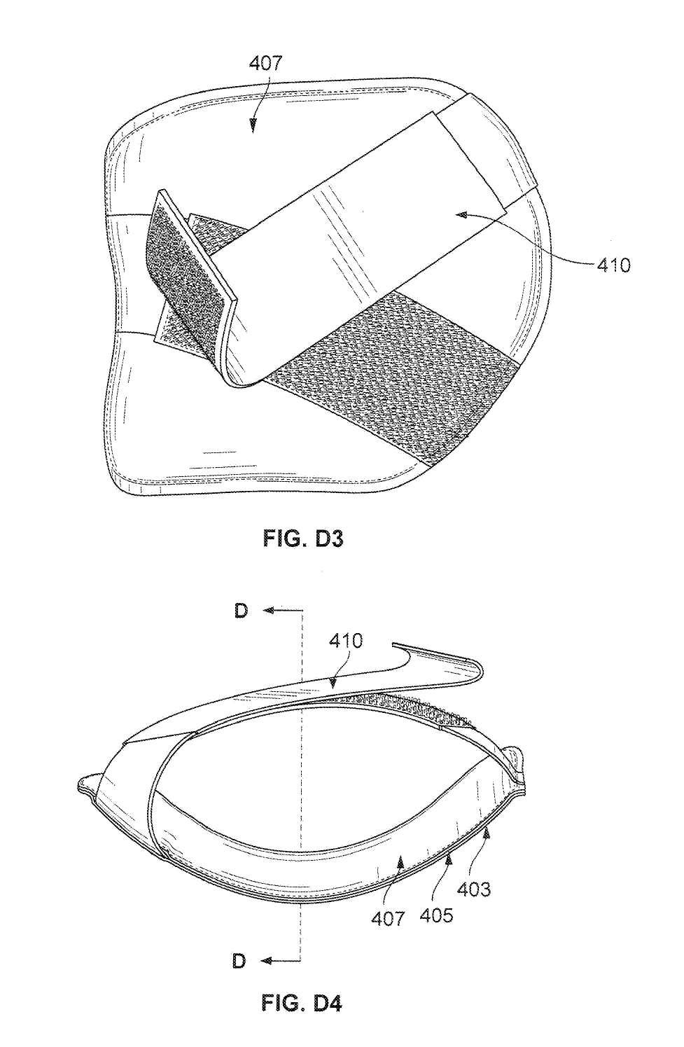

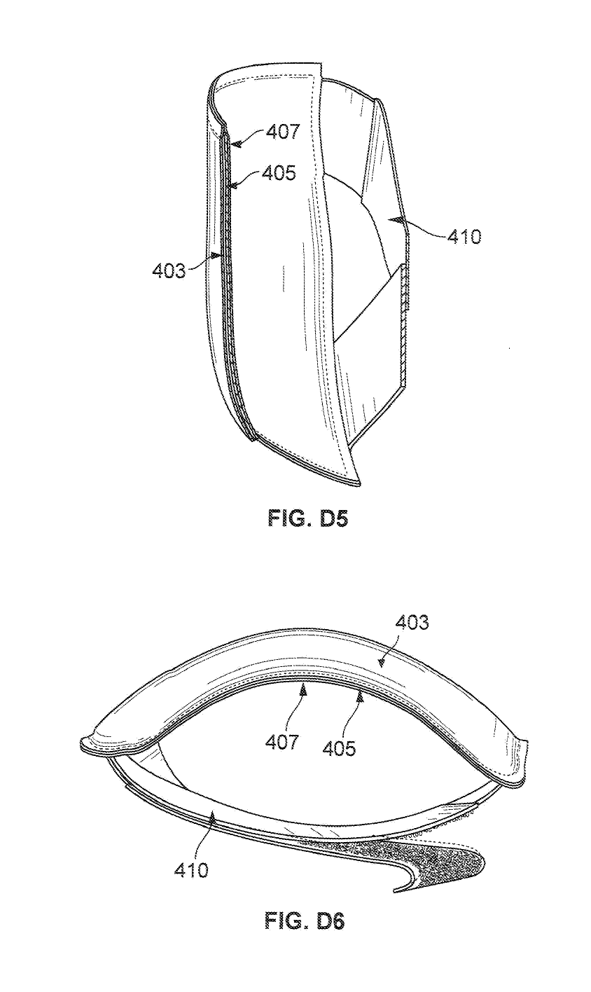

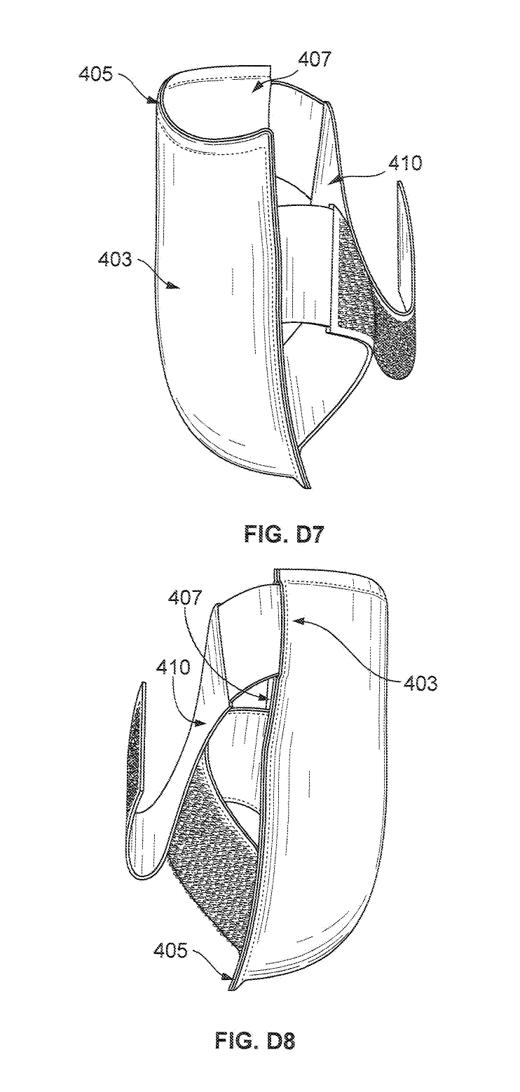

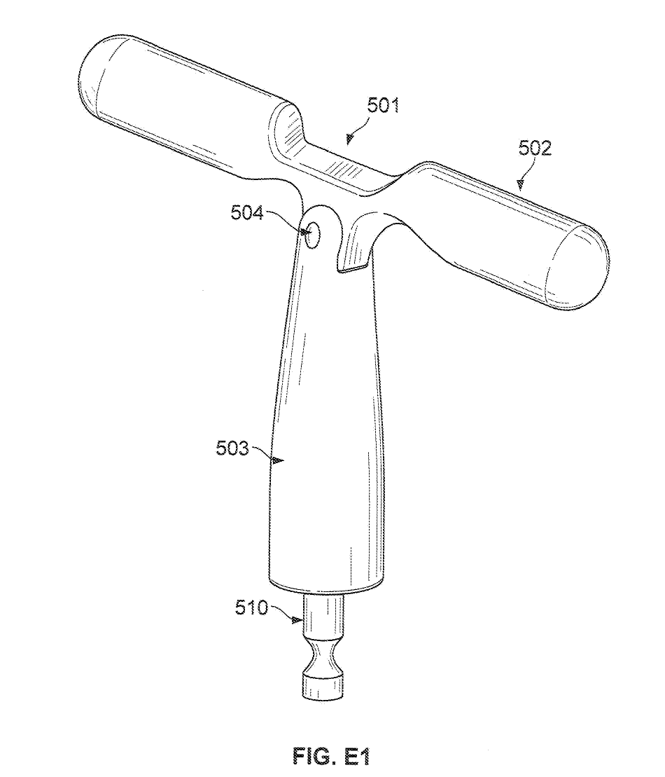

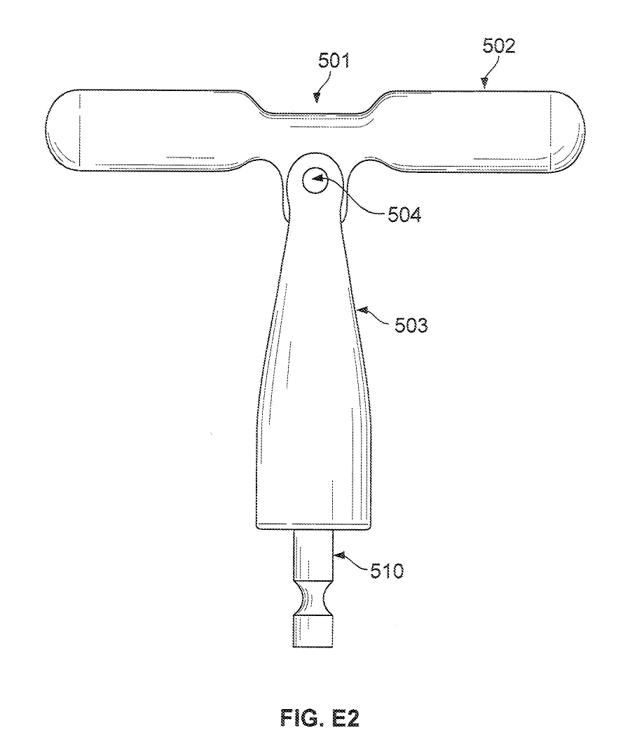

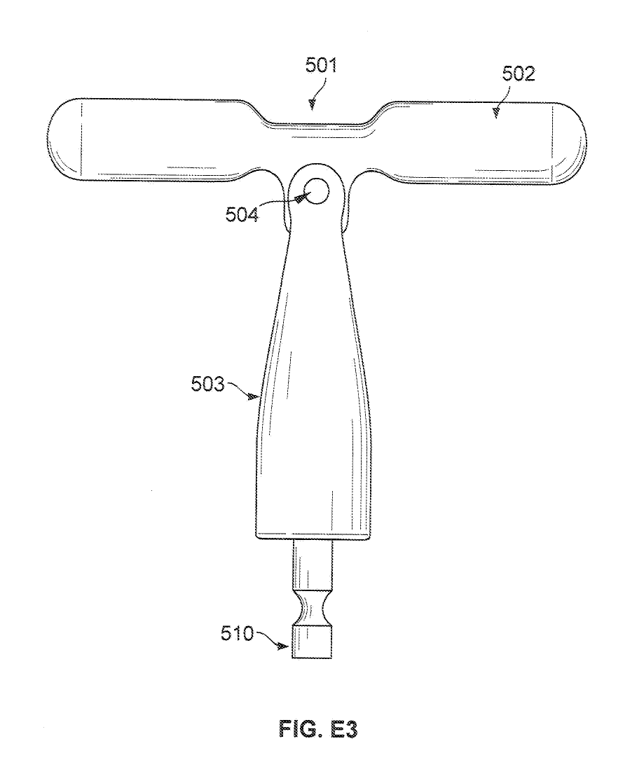

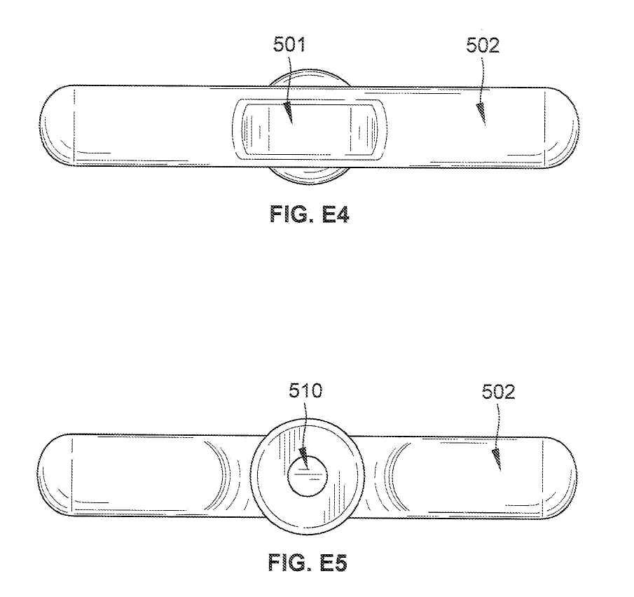

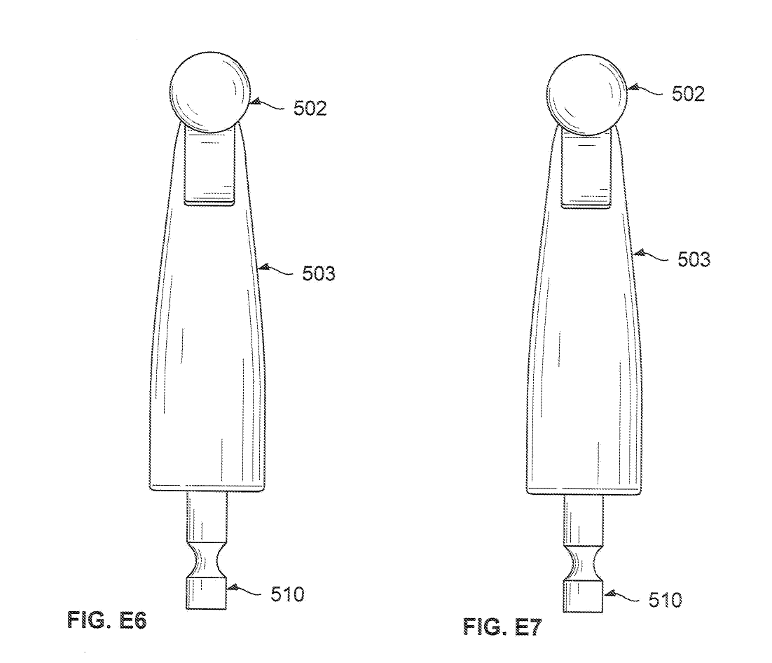

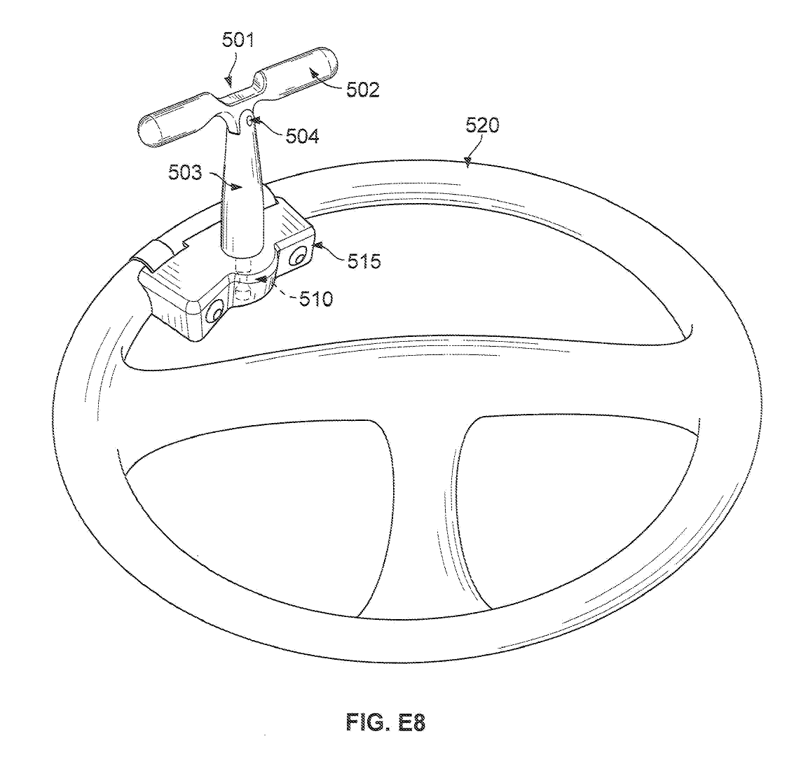

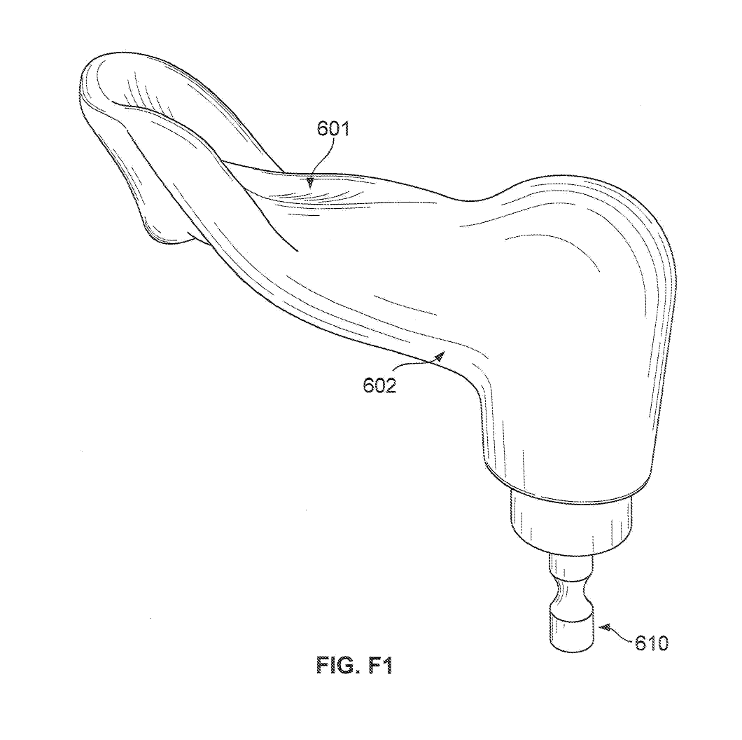

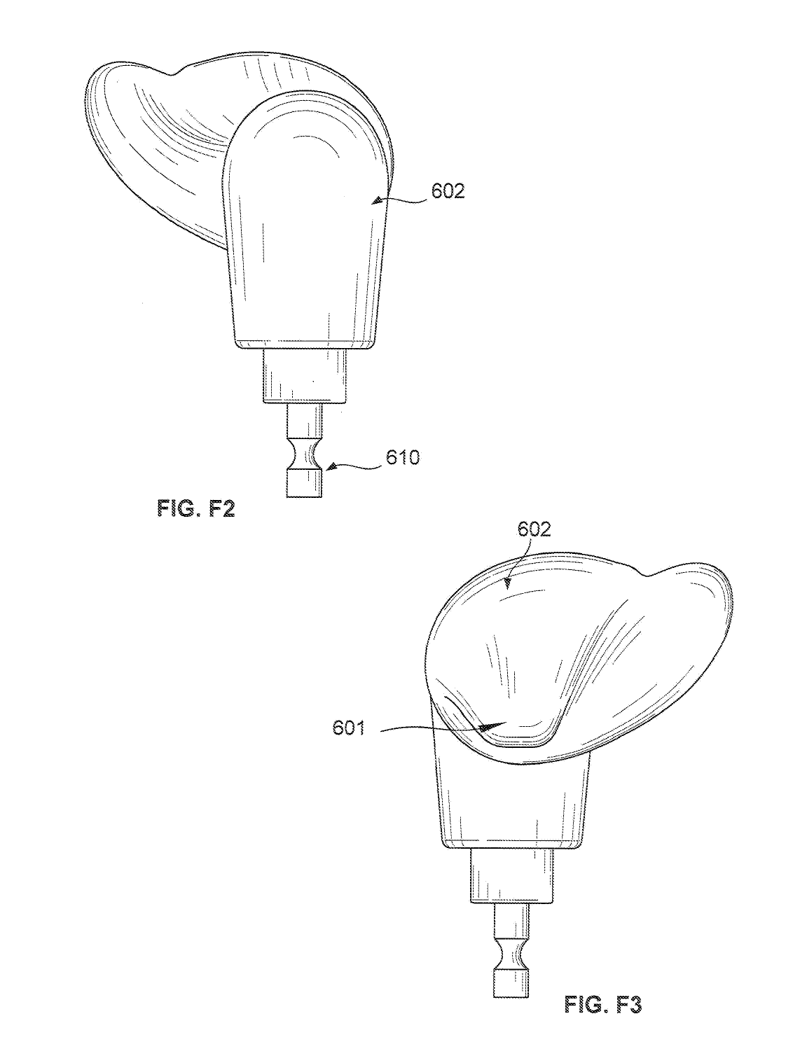

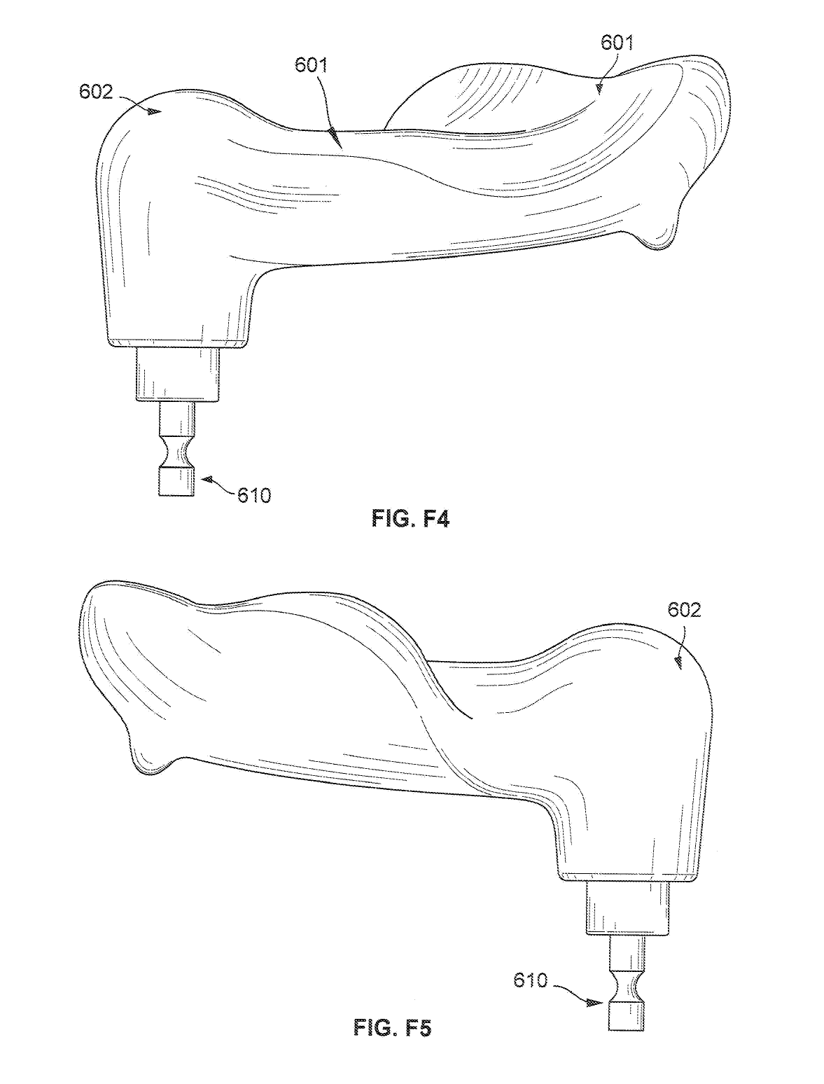

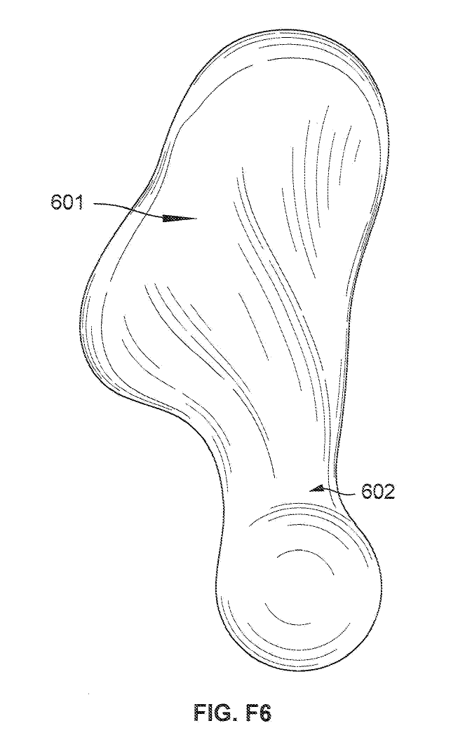

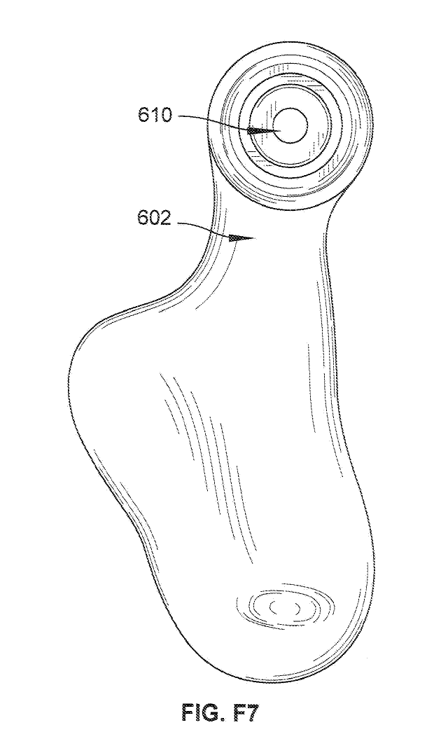

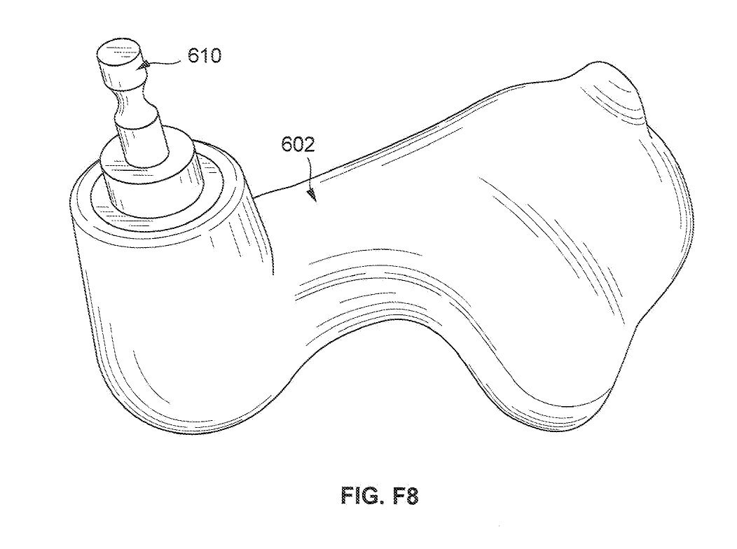

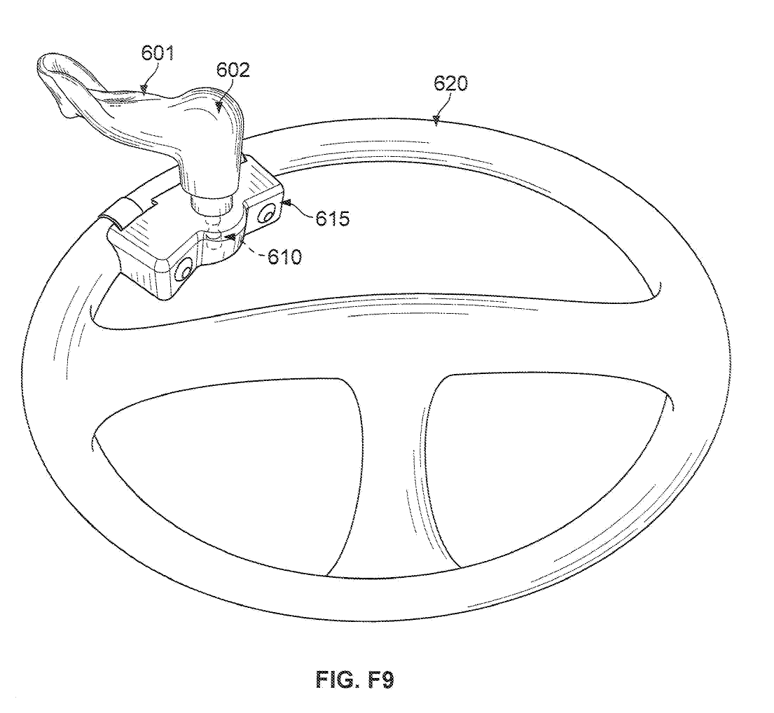

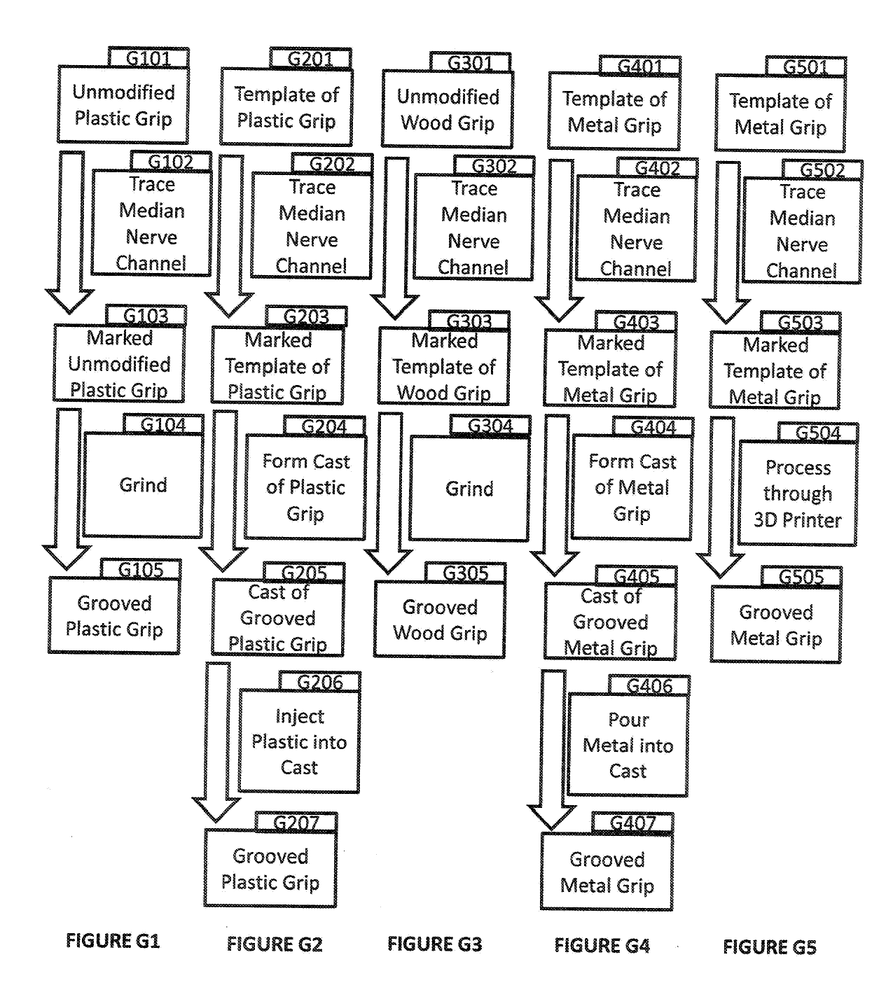

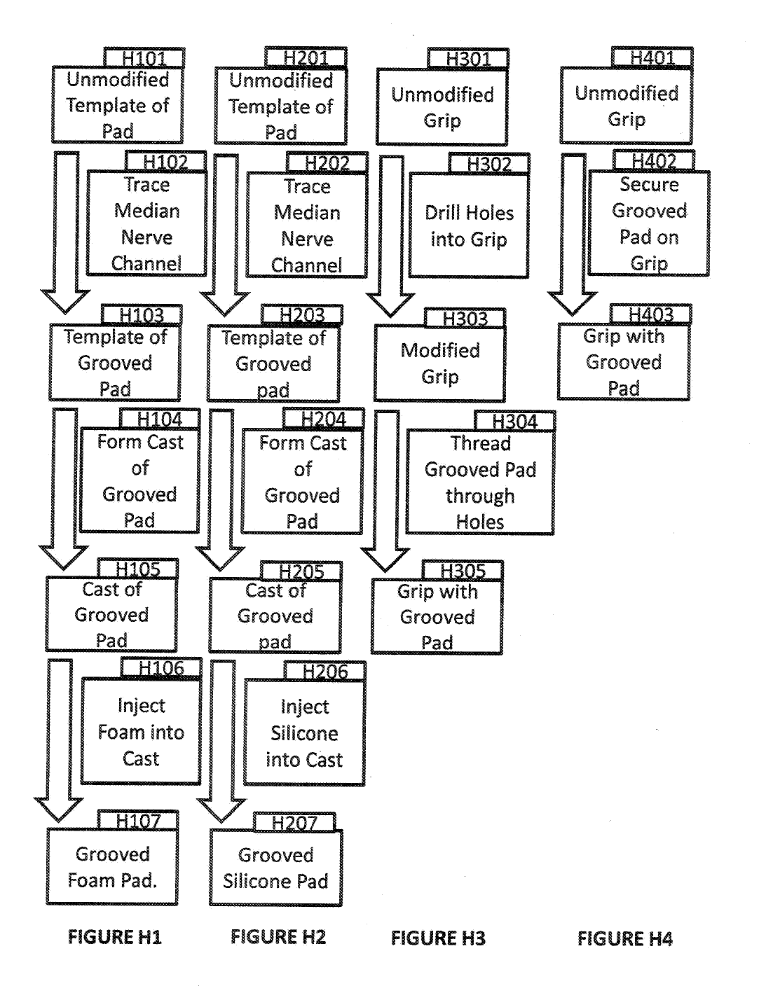

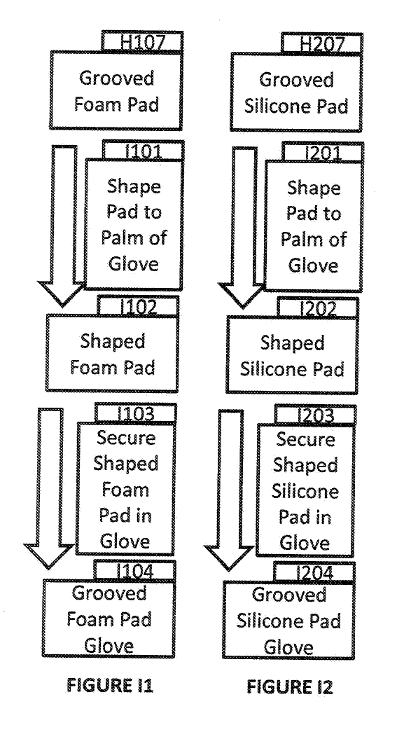

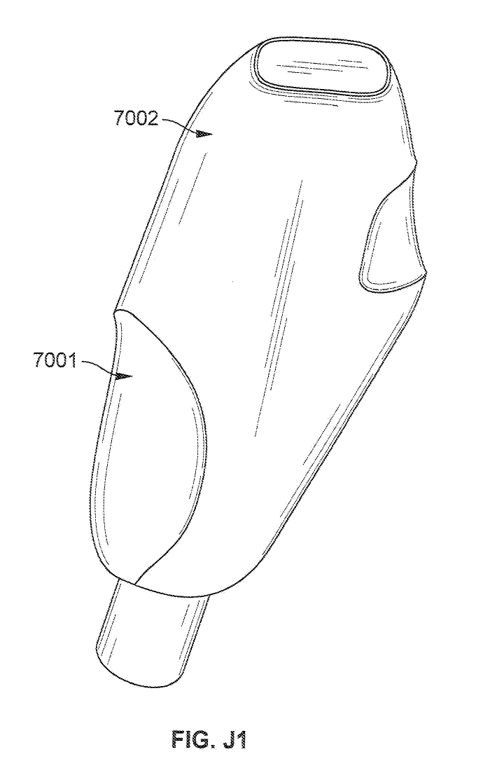

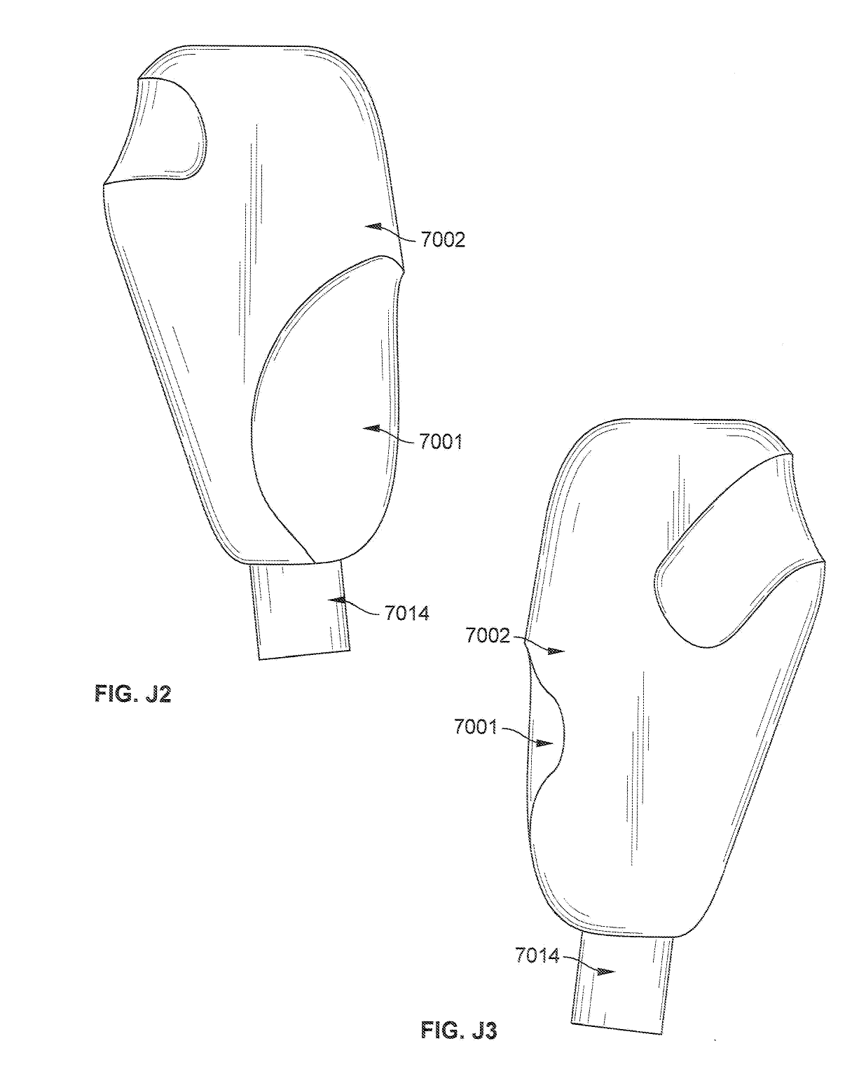

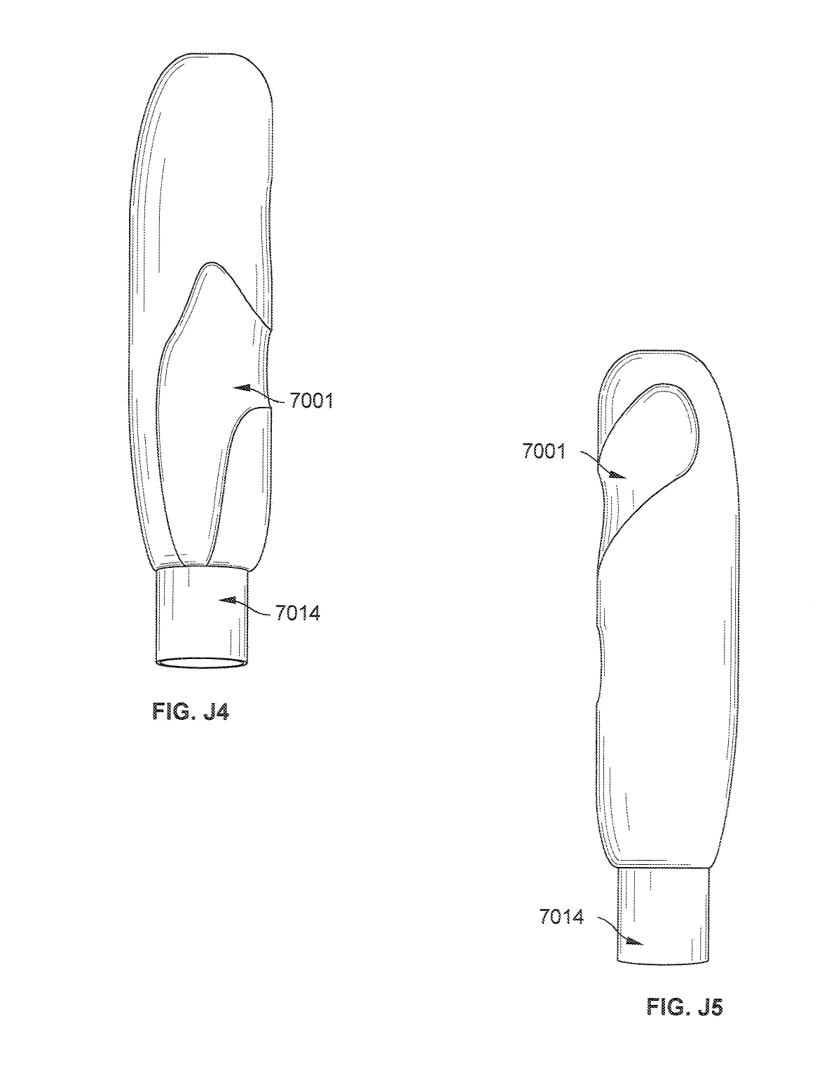

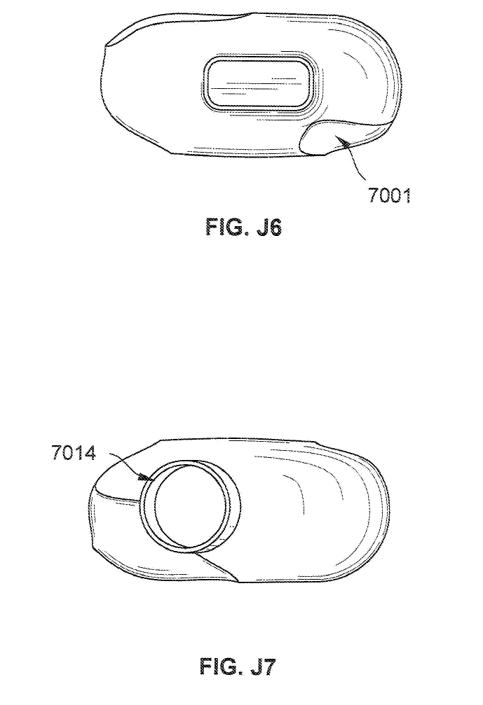

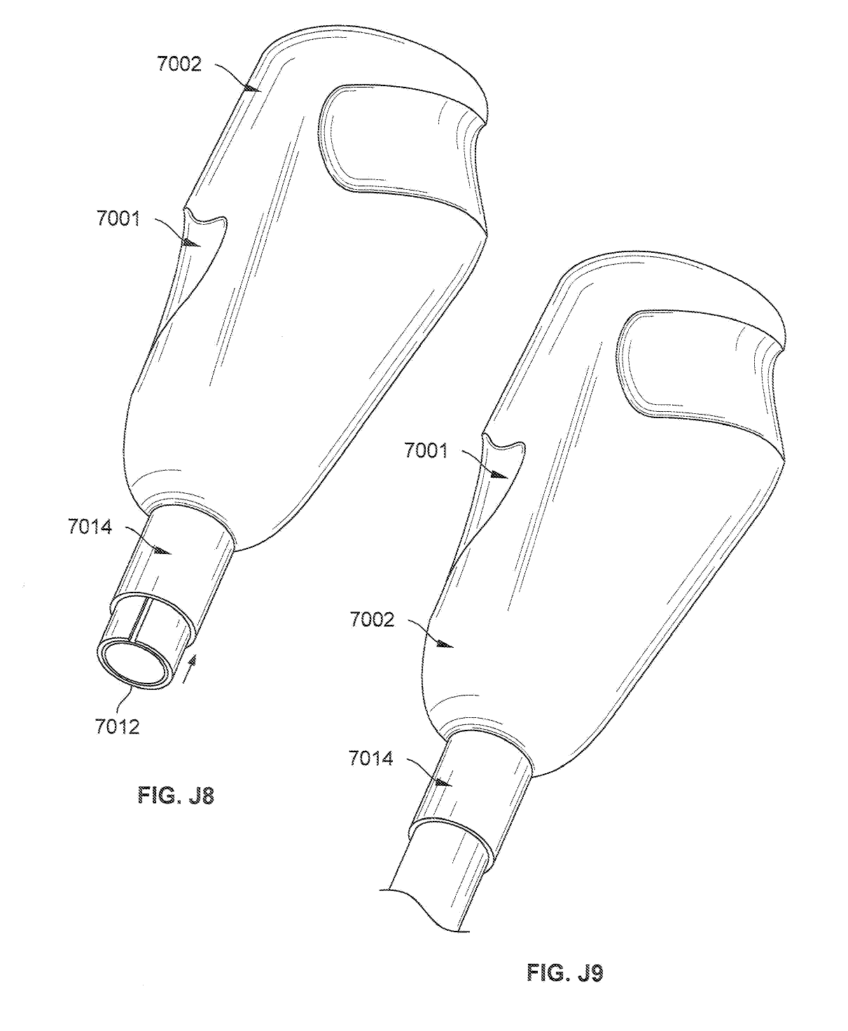

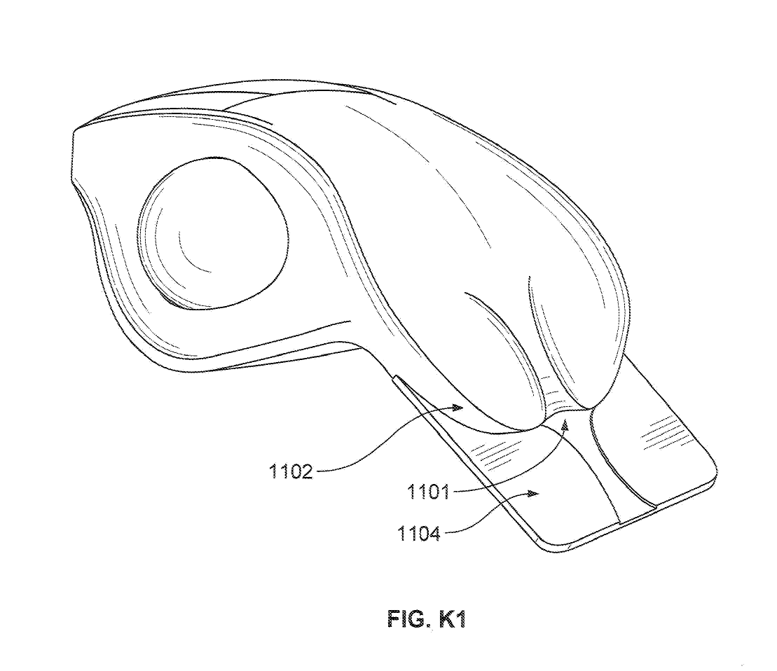

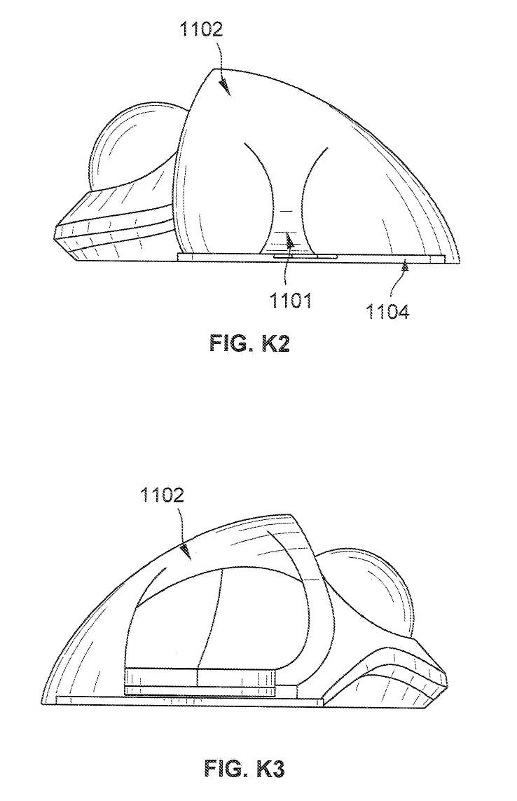

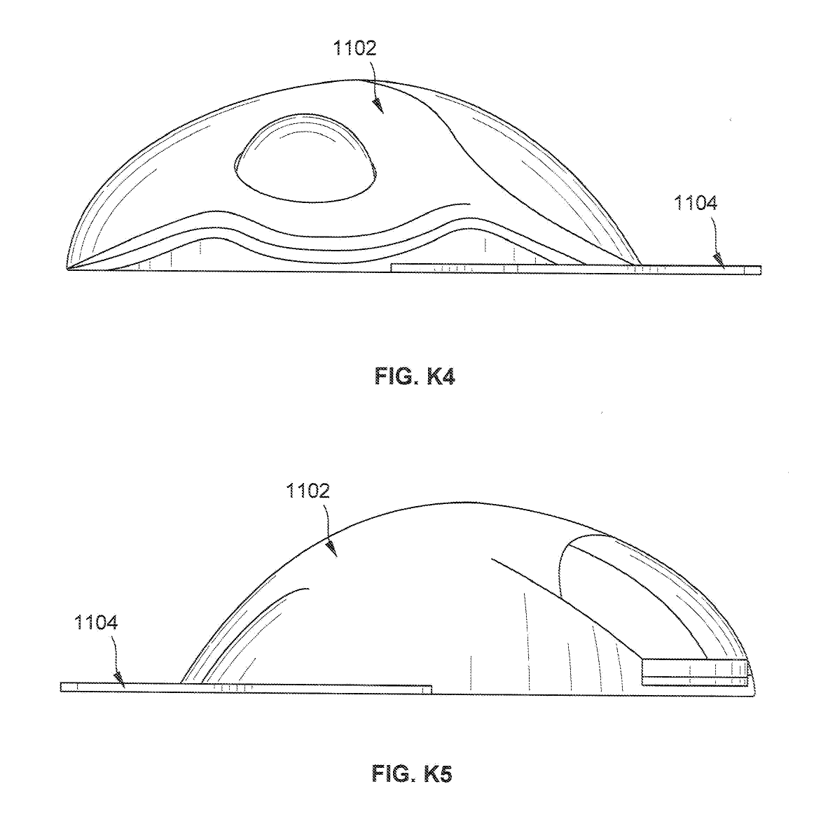

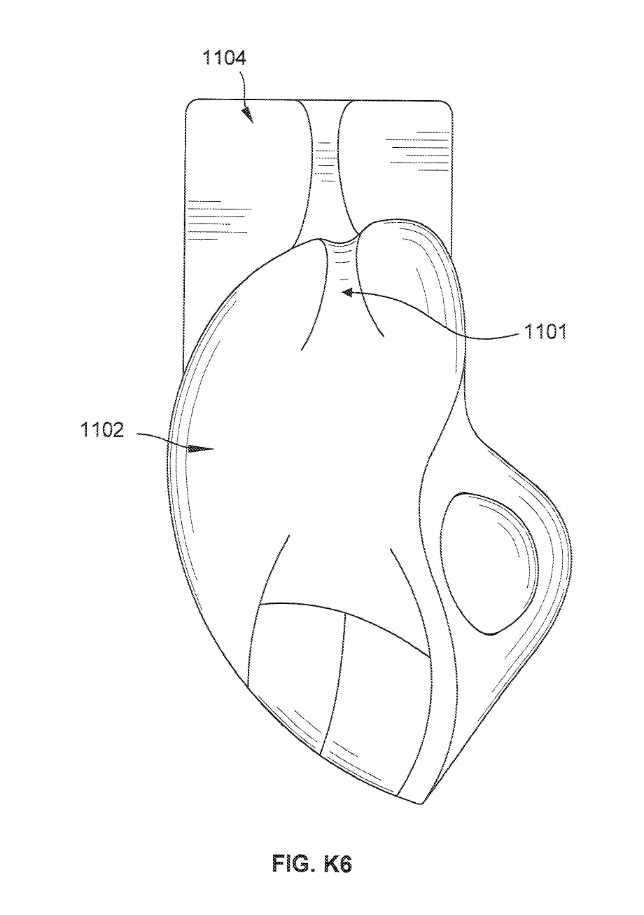

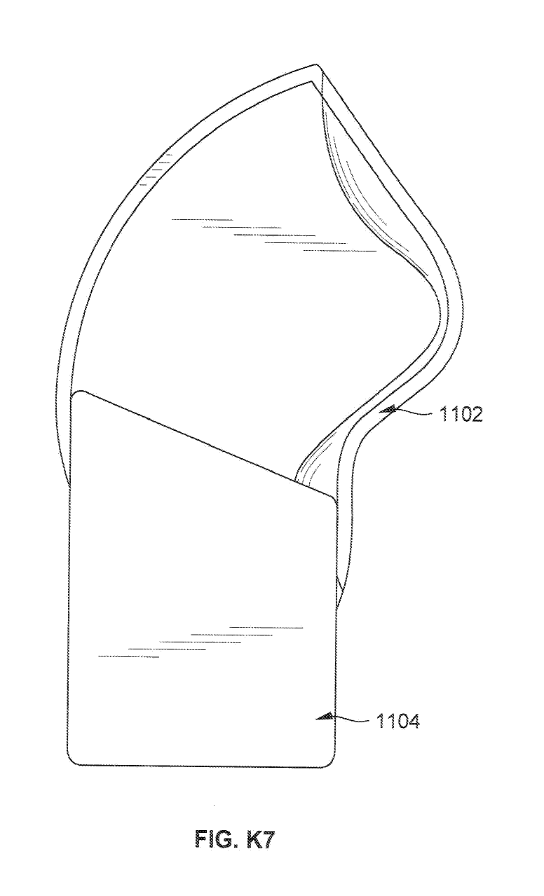

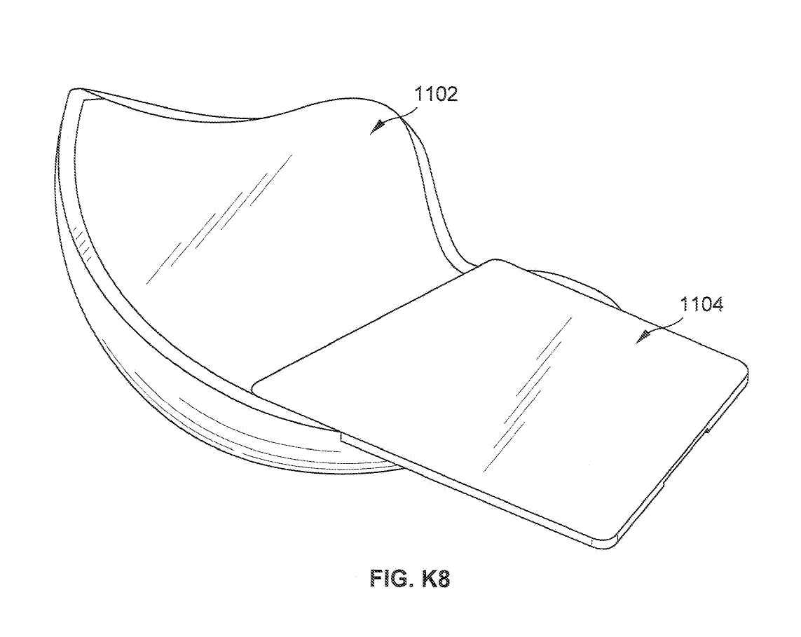

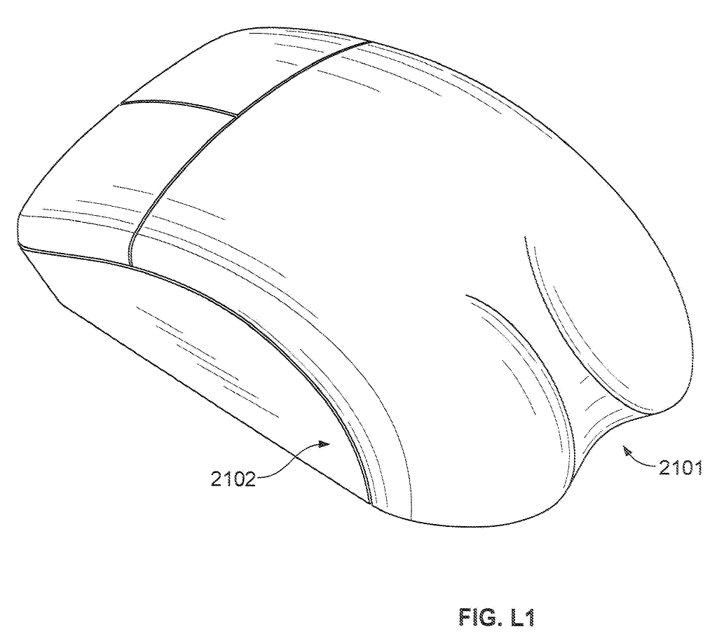

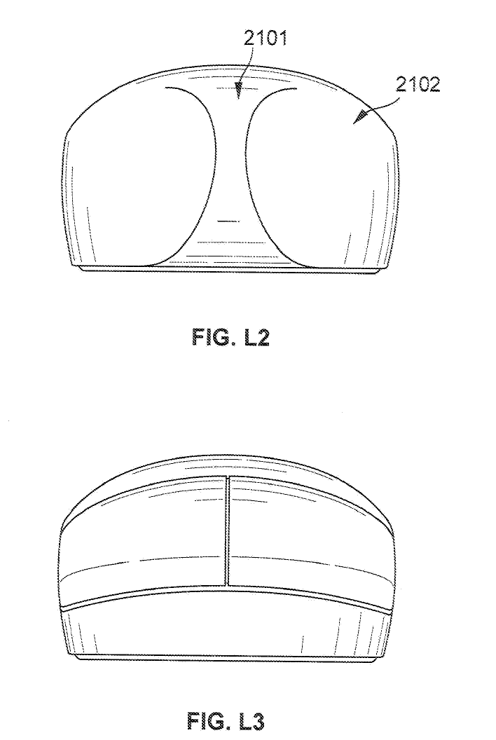

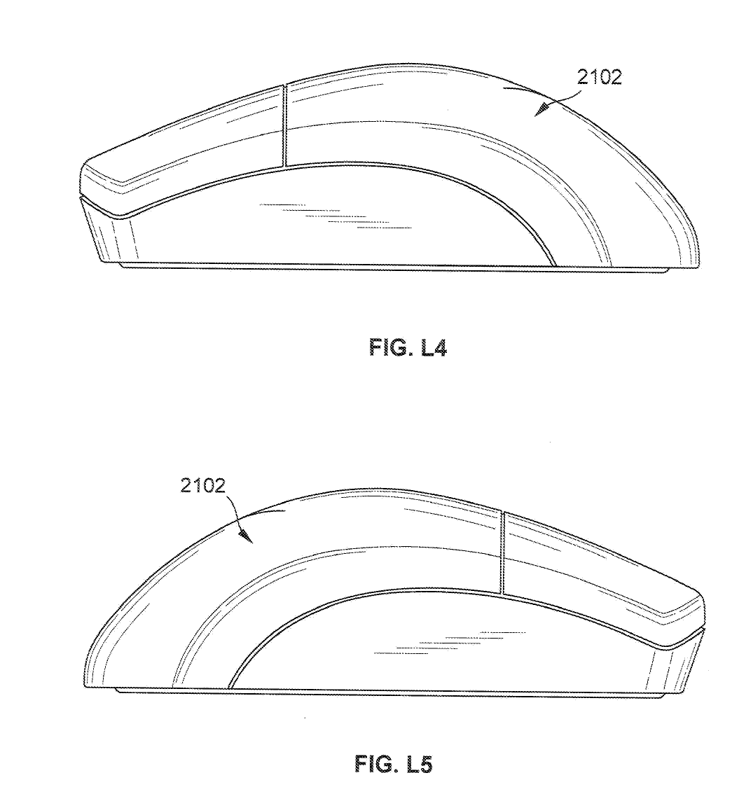

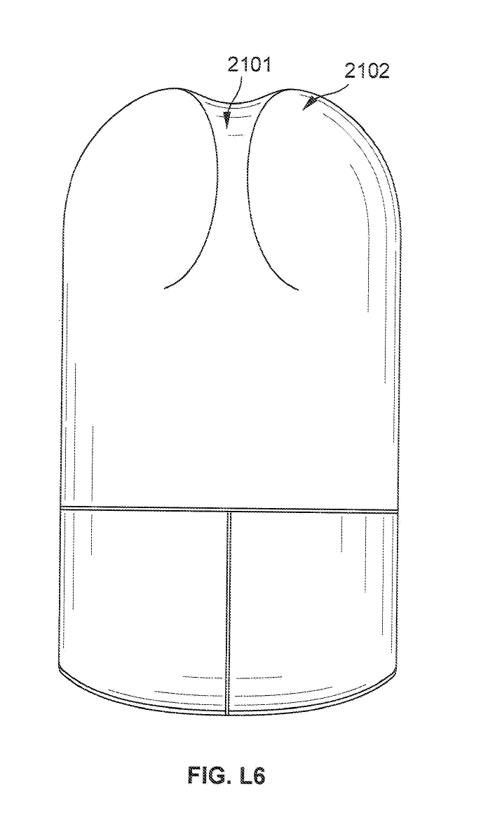

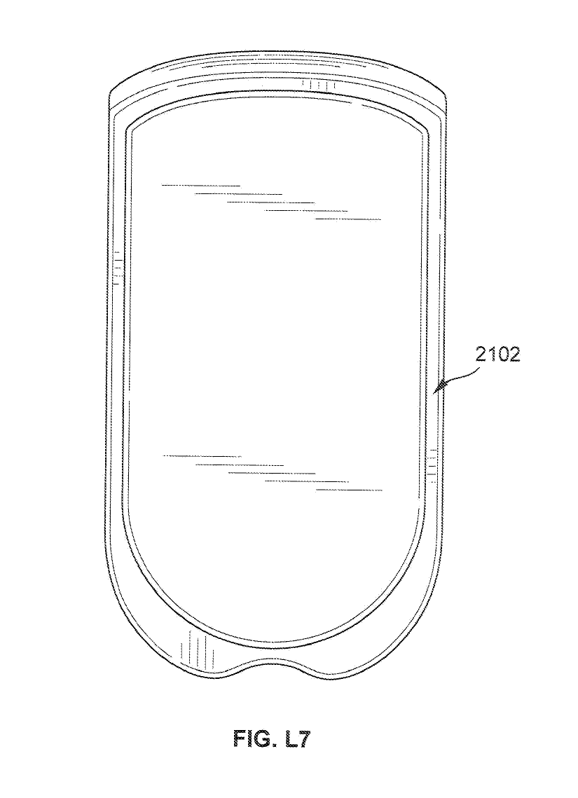

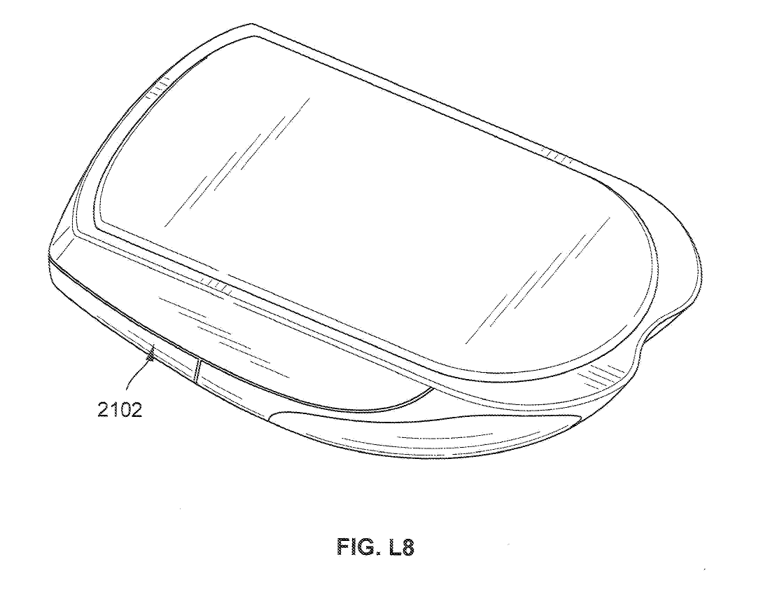

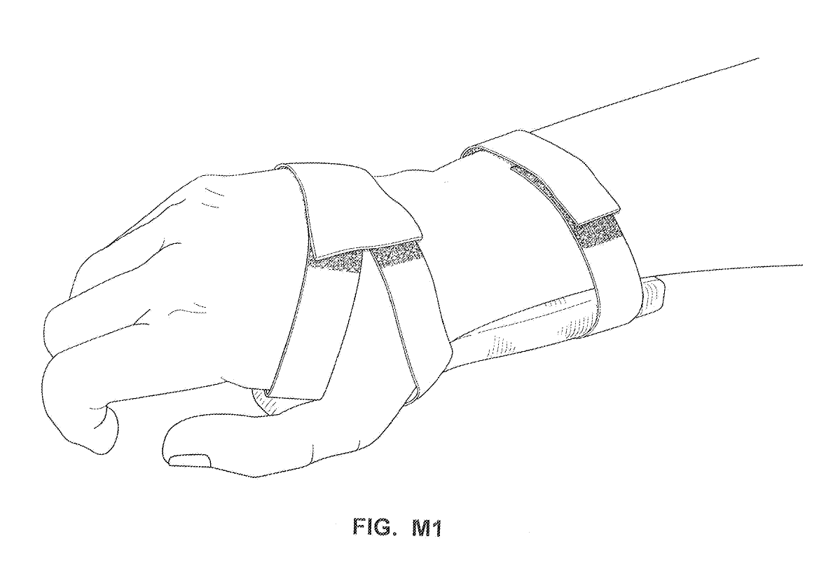

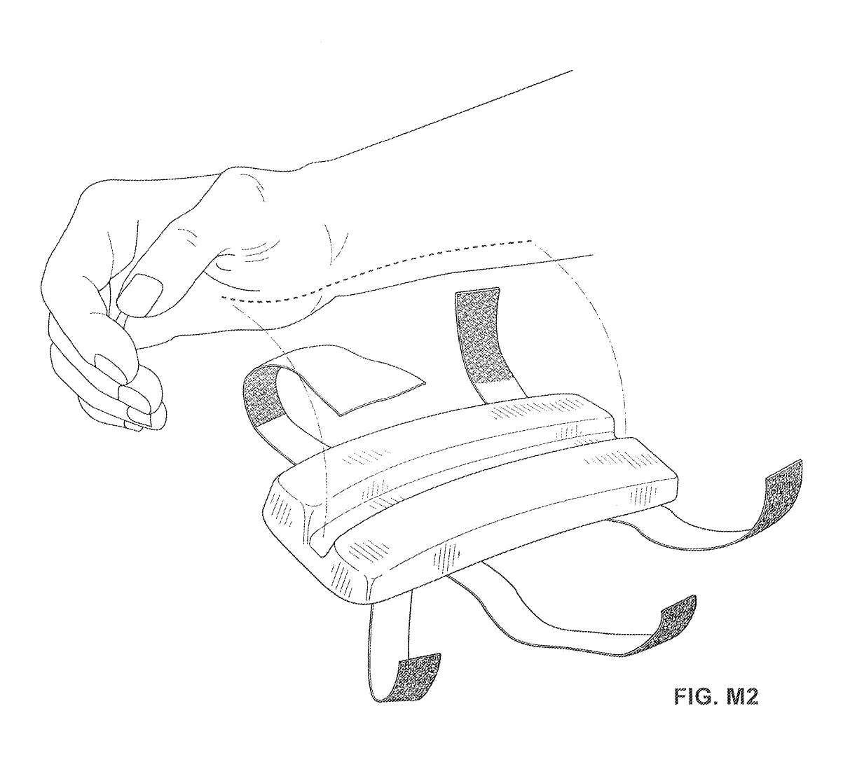

An alternative embodiment of the grooved grip is illustrated in FIGS. D1-D8. In FIG. D2, a grooved glove 402 may have an outer layer 403. In FIG. 104, a grooved glove may have an inner layer 407. In FIG. D6, a grooved pad 405 may lie between outer layer 403 and inner layer 407. The grooved pad 405 may have a channel configured to relieve pressure on a median nerve on a palmar surface on a hand. In the embodiment, the grooved pad may be stitched between outer layer 403 and inner layer 407. In the embodiment outer layer 403 and inner layer 407 may be a pebbled rubber material. However, outer layer 403 and inner layer 407 may comprise any other type of material that supports grooved pad 405, such as a textile material. In the embodiment, grooved glove 402 may be secured to a hand using a Velcro strap 410 affixed to inner layer 407. However, grooved glove 402 may be secured to a hand using any other material that secures grooved pad 405 to the palmar surface of a hand.

FIGS. E1-E8 show views of a grooved pivoting steering wheel spinning grip, according to an embodiment. In FIG. E1, a head 502 may have a channel 501 configured to relieve pressure on a median nerve on a palmar surface of a hand. A pivot joint 504 may be a socket and pin joint that allows movement of the head 502 perpendicular to the body 503. A body 503 may have a mounting pin 510. In FIG. E8, a steering device 520 may be connected to the mounting pin 510 using a mounting device 515. FIGS. F1-F9 show views of a left-handed grooved steering wheel spinner grip. According to an embodiment, in FIG. F1, a head 602 may have a channel 601 configured to relieve pressure on a median nerve on a palmar surface of a hand. A mounting pin 610 may be connected to a steering device 620 using a mounting device 615, as shown in FIG. F9.

An embodiment of a grooved grip, a left-handed grooved pad, is shown in FIGS. J1-J9. (Note that a right-handed grooved pad, which would be an opposite of the pad in FIGS. J1-J9, can also be used.) In the embodiment, the grooved pad may be foam. However, the grooved pad may comprise any other material that can be configured into a grooved pad, such as silicone. In FIG. J4, the embodiment may comprise a body 7002 with a channel 7001 configured to relieve pressure on a median nerve on a palmar surface on a hand. In the embodiment in FIG. J8, the bottom surface of the grooved pad may terminate at a mounting hole 7014 configured to receive a grip 7012 or the like. Alternatively, the embodiment may be attached to a grip with an adhesive, such as an anaerobic adhesive, cyanoacrylate adhesive, toughened acrylic, silicone adhesive, polyurethane adhesive, phenolic, polyimide adhesive, plastisol adhesive, polyvinyl acetate adhesive, pressure-sensitive adhesive, or any other adhesive suitable to secure a grooved pad to a grip. In the embodiment in FIG. J9, mounting hole 7014 may be attached to bicycle handle. While the embodiment in FIG. J9 is illustrated as attaching to a bicycle handle grip, the embodiment may be attached to any structure configured to receive the embodiment, including a grip, steering wheel spinner grip, cane handle, machine (e.g., lawnmower handle), crutch handle, exercise and/or sporting equipment (e.g., hand-cycle handle, bicycle pump), tools (e.g., electric drill handle, impact wrench handle, lever, hammer handle, or sledgehammer handle).

An embodiment of a grooved grip, a grooved computer trackball, is illustrated in FIGS. K1-K8. In FIG. K2, a grooved trackball configured for a left hand may have a body 1102. In the embodiment, the body 1102 may be plastic. However, the body 1102 may comprise any other material that can be configured into a grooved trackball, such as wood or metal. Along the back surface of body 1102 as seen in FIG. K6 may be a channel 1101 configured to relieve pressure on a median nerve on a palmar surface on a hand. Alternatively, as seen in FIG. K7, the inferior surface of body 1102 may comprise a grooved attachment 1104 with a channel configured to relieve pressure on a median nerve on a palmar surface on a hand. In FIG. K8, the grooved attachment 1104 may be plastic. However, the grooved attachment 1104 may comprise any other material that can be configured into a grooved attachment, such as wood, metal, foam, or silicone. In the embodiment, the grooved attachment 1104 may be attached to body 1102 using an adhesive. However, grooved attachment 1104 may be attached to body 1102 using any process suitable to attach grooved attachment 1104 to body 1102. While the embodiment in FIG. K1 illustrates a grooved attachment 1104 attached to a computer trackball, grooved attachment 1104 may be attached to any structure configured to receive it, such as a computer keyboard, computer mouse, or computer mousepad. (Note that the trackball may he left-handed, right-handed or ambidextrous.)

An embodiment of a grooved computer mouse is illustrated in FIGS. L1-L8. In FIG. L2, a grooved mouse configured for a left hand may have a body 2102. Along the back surface of body 2102 as seen in FIG. L6 may be a channel 2101 configured to relieve pressure on a median nerve on a palmar surface on a hand. While the embodiment in FIG. L1 illustrates a channel 2101 on a grooved computer mouse, channel 2101 may be configured for use on a computer keyboard. Note that the mouse may be left-handed, right-handed or ambidextrous.

An embodiment of a grooved splint is illustrated in FIG. M1, according to an embodiment. FIG. M2 illustrates an inside view of the grooved splint. A groove is illustrated in FIG. M2. The groove carries up the forearm and the height puts the wrist in a neutral position. The grooved splint may be made of silicone and may be formed with a CMC.

As shown in FIGS. G1-G5, a grooved grip may be formed from a variety of materials, including plastic, wood, or metal. While a grip is referred to in the following examples, either a grip or a steering wheel grip may be formed using the methods G1-G5, or any other method disclosed in this application or known in the art. In addition, the methods of G1-G2 may be used to form a mouse or trackball. Of course, any other methods disclosed in this application or known in the art may also be used.

In FIG. G1, a grooved plastic grip may be formed through modification of a pre-existing unmodified plastic grip G101. Unmodified plastic grip G101 may comprise polystyrene, polyvinyl chloride, high-density polyethylene, low-density polyethylene, acrylonitrile butadiene styrene, polycarbonate, polyurethane, rubber, or synthetic rubber, or any other plastic suitable to serve as a grip configured to be grasped by a hand. In G102, a channel configured to relieve pressure on a median nerve on a palmar surface on a hand may be traced onto unmodified plastic grip G101 to form a marked unmodified plastic grip G103. In G104, a channel configured to relieve pressure on a median nerve on a palmar surface may be winded into marked unmodified plastic grip G103 to form grooved plastic grip G105. A rotary tool, such as a lathe, or a filing tool may be used to form the channel.

Alternatively, as shown in FIG. G2, a channel configured to relieve pressure on a median nerve on a palmar surface on a hand may be introduced to a plastic grip utilizing an injection molding process. In G201, a template of a plastic grip configured to be grasped by a hand may be created. In G202, a channel configured to relieve pressure on a median nerve on a palmar surface on a hand may be traced onto a template of plastic grip G201 to create a marked template of a plastic grip G203. In G204, a cast of a plastic grip G205 may be formed from a material such as clay or any other material suitable to serve as a cast for plastic injection molding. In G206, molten plastic material, such as polystyrene, polyvinyl chloride, high-density polyethylene, low-density polyethylene, acrylonitrile butadiene styrene, polycarbonate, polyurethane, rubber, or synthetic rubber, or any other plastic suitable to serve as a grip configured to be grasped by a hand, may be injected into a cast of a grooved plastic grip G205. The plastic material may be allowed to solidify into a grooved plastic grip G207 and removed from a east of grooved plastic grip G205.

As shown in FIG. G3, a channel configured to relieve pressure on a median nerve on a palmar surface on a hand may be introduced to a unmodified wood grip G301. Unmodified wood grip G301 may comprise softwoods—cedar, fir, pine, or redwood—or hardwoods—ash, birch, cherry, mahogany, maple, oak, poplar, teak, or walnut—or any other wood suitable to form a grip configured to be grasped by a hand. In G302, a channel configured to relieve pressure on a median nerve on a palmar surface on a hand may be traced onto an unmodified wood grip G301 to form a marked template of wood grip G303. In G304, a channel configured to relieve pressure on a median nerve on a palmar surface may be grinded into marked template of wood grip G303 to form grooved wood grip G305. A rotary tool, such as a lathe, computerized milling machine (CMC) or a filing tool may be used to form the channel. (Note that the CMC may be used for wood, metal or plastic.)

As shown in FIG. G4, a channel configured to relieve pressure on a median nerve on a palmar surface on a hand may be introduced to a metal grip utilizing a metal casting process. A template of a metal grip G401 configured to be grasped by a hand may be created. In G401, a channel configured to relieve pressure on a median nerve on a palmar surface on a hand may be traced onto a template of a metal grip G401 to form a marked template of a metal grip G403. In G404, a cast of a grooved metal grip G405 may be formed from a material such as plaster, wax, clay, sand or any other material suitable to serve as a cast for metal casting. In GB406, a grooved metal grip G407 may be formed by pouring a molten metal material, such as zinc, lead, copper, silver, tin, aluminum, iron, or any other metal suitable to serve as a grip configured to be grasped by a hand, into a cast of a grooved metal grip G405. Grooved metal grip G407 may be allowed to solidify and may be removed from a cast of a grooved metal grip G405.

As shown in FIG. G5, a channel configured to relieve pressure on a median nerve on a palmar surface on a hand may be introduced to a metal grip utilizing 3D printing. A template of a metal grip G501 configured to be grasped by a hand may be created utilizing computer aided design programs or animation modeling software. In G502, a median nerve channel measured to conform to a median nerve on a palmar surface on a hand G502 may be incorporated into a template of a metal grip G501 to create a marked template of a metal grip G503. In G504, a grooved metal grip G505 may be created by processing a marked template of a metal grip through a 3D printer. The 3D printer may utilize 3D printing technology, such as fused deposition modeling, electron beam freeform fabrication, direct metal laser sintering, electron beam melting, selective laser sintering, laminated object manufacturing, or any other 3D printing technology capable of creating a metal grip configured to be grasped by a hand.

FIGS. H1-H4 illustrate example processes of manufacturing pads. These processes, as well as any other methods disclosed in this application or known in the art may be used to form a pad or a splint.

As shown in FIG. H1, a channel configured to relieve pressure on a median nerve on a palmar surface on a hand may be introduced to a foam pad utilizing an injection molding process. In H101 an unmodified template of a pad may be created. In H102, a channel configured to relieve pressure on a median nerve on a palmar surface on a hand may be traced onto an unmodified template of a pad H101 to create a template of a grooved pad H103. In H104, a cast of a grooved pad H105 may be formed from a material such as clay or any other material suitable to serve as a cast for foam injection molding. In H106, foam material, such as polyurethane, high density foam, evlon, high resilience foam, latex rubber foam, supreem, rebond, viscolastic foam, closed cell foam, dry fast foam, or any other foam material suitable to be configured to be grasped by a hand may be injected into a cast of a grooved pad H105. The foam material may be allowed to solidify into a grooved foam pad H107 and may be removed from the cast of grooved pad H105.

As shown in FIG. H2, a channel configured to relieve pressure on a median nerve on a palmar surface on a hand may be introduced to a silicone pad utilizing an injection molding process. In H201, an unmodified template of a pad may be created. In H202, a channel configured to relieve pressure on a median nerve on a palmar surface on a hand may be traced onto an unmodified template of a pad H201 to create a template of a grooved pad H203. In H204, a cast of a grooved pad H205 may be formed from a material such as clay or any other material suitable to serve as a cast for foam injection molding. In H206, silicone material, such as liquid silicone rubber, or any other silicone material suitable to be configured to be grasped by a hand may be injected into a cast of a grooved pad H205. The silicone material may be allowed to solidify into a grooved silicone pad H207 and may be removed from the cast of grooved pad H205.

As shown in FIG. H3, a channel configured to relieve pressure on a median nerve on a palmar surface on a hand may be introduced to a grip with a grooved pad. In H302, a hole may be drilled into an unmodified grip H301 to form a modified grip H303. In H304, a grip with a grooved pad H305 may be formed by attaching the grooved pad comprising a thread to a modified grip H303. A grooved pad may be attached to a modified grip H303 by threading the grooved pad through a hole drilled into a modified grip H303.

As shown in FIG. H4, a channel configured to relieve pressure on a median nerve on a palmar surface on a hand may be introduced to a grip with a grooved pad using an adhesive. In H401, an unmodified grip may be created. In H402, a grip with a grooved pad H403 may be created by securing a grooved pad onto an unmodified grip H401 using an adhesive, such as an anaerobic adhesive, cyanoacrylate adhesive, toughened acrylic, silicone adhesive, polyurethane adhesive, phenolic, polyimide adhesive, plastisol adhesive, polyvinyl acetate adhesive, pressure-sensitive adhesive, or any other adhesive suitable to secure a grooved pad to an unmodified grip H401.

FIGS. I1-I2 illustrate example processes of manufacturing a glove. These processes, or any other methods disclosed in this application or known in the art, may be used to form a glove or a splint.

As shown in FIG. I1, a channel configured to relieve pressure on a median nerve on a hand may be introduced to a glove with a grooved foam pad. In I101, a shaped foam pad I102 may be formed by shaping a grooved foam pad H107. The shaped foam pad I102 may be shaped to conform to a glove during or after the injection molding process illustrated in FIG. H1. In I103, a grooved foam pad glove I104 may be created by securing a shaped foam pad I102 to a glove. A shaped foam pad I102 may be secured to a glove by stitching the pad to the glove or by an adhesive, such as an anaerobic adhesive, cyanoacrylate adhesive, toughened acrylic, silicone adhesive, polyurethane adhesive, phenolic, polyimide adhesive, plastisol adhesive, polyvinyl acetate adhesive, pressure-sensitive adhesive, or any other adhesive suitable to secure a shaped foam pad I102 to a glove.

As shown in FIG. I2, a channel configured to relieve pressure on a median nerve on a hand may be introduced to a glove with a grooved silicone pad. (Note that the glove may be left-handed or right-handed.) In I201, a shaped silicone pad I202 may be formed by shaping a grooved silicone pad H207. The shaped silicone pad I202 may be shaped to conform to a glove during or after the injection molding process illustrated in FIG. H2. In I203, a grooved silicone pad glove I204 may be created by securing a shaped silicone pad I202 to a glove. A shaped silicone pad I202 may be secured to a glove by stitching the pad to the glove or by an adhesive, such as an anaerobic adhesive, cyanoacrylate adhesive, toughened acrylic, silicone adhesive, polyurethane adhesive, phenolic, polyimide adhesive, plastisol adhesive, polyvinyl acetate adhesive, pressure-sensitive adhesive, or any other adhesive suitable to secure a shaped silicone pad I202 to a glove.

In this specification, “a” and “an” and similar phrases are to be interpreted as at least one” and “one or more.” References to “an” embodiment in this disclosure are not necessarily to the same embodiment.

In addition, the terms “including” and “comprising” should be interpreted as “including, but not limited to”.

The invention may be embodied in other specific forms without departing from the spirit or essential characteristics thereof. For example, while left-handed or right-handed embodiments may be described, it should be understood that the opposite and also ambidextrous embodiments are also intended to be embraced in this application. In addition, while an ambidextrous embodiments may be described, it should be understood that right-handed or left-handed embodiments are also intended to be embraced in this application. The embodiment described above is therefore to be considered in all respects as illustrative and not restrictive, the scope of the invention being indicated by the hereafter appended claims rather than by the foregoing description, and all changes which conic within the meaning and range of equivalency of the claims are therefore intended to be embraced therein.

Further, the purposes of the Abstract of the Disclosure is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract of the Disclosure it not intended to be limiting as to the scope in any way.

Finally, it is the applicant's intent that only claim that include the express language “means for” or “step for” be interpreted under 35 U.S.C. §112(f). Claims that do not expressly include the phrase “means for” or “step for” are not to be interpreted under 35 U.S.C. §112(f).

What is claimed is:

1. An apparatus comprising:
a grip for receiving a hand;
the grip having a first side and a second side; and
a recess formed between the first side and the second side, the recess having a shape corresponding to a median nerve on a palmar surface of a hand so that pressure on a median nerve is relieved.

2. The apparatus of claim 1, wherein the grip further comprises an indentation configured to prevent slippage of the index finger of the users hand.

3. The apparatus of claim 1, wherein the grip is made of or further comprises padding.

4. The apparatus of claim 1, wherein the grip is configured to support a left hand, configured to support a right hand, or ambidextrous.

5. The apparatus of claim 1, wherein the grip has the shape of a T-handle or an L-handle.

6. The apparatus of claim 1, wherein the grip comprises a pivot so that the grip is able to be adjusted relative to a grip attachment.

7. The apparatus of claim 1, wherein the grip is configured to attach to medical equipment.

8. The apparatus of claim 1, wherein the grip is configured to attach to a tool.

9. The apparatus of claim 1, wherein the grip is configured to attach to sporting equipment.

10. The apparatus of claim 1, wherein the grip is configured to attach to machinery.

11. The apparatus of claim 1, wherein the grip is configured to attach to a steering wheel.

12. The apparatus of claim 1, wherein the grip is configured to attach to a steering wheel by a quick-release mechanism.

13. The apparatus of claim 1, wherein the grip comprises a pivot so that the grip is adjustable with respect to the steering wheel.

14. An apparatus comprising:
a computer mouse for receiving a hand;
the computer mouse having a first side and a second side; and
a recess formed between the first side and the second side, the recess having a shape corresponding to a median nerve on a palmar surface of a hand so that pressure on a median nerve is relieved.

15. An apparatus comprising:
a splint for receiving a hand;
the splint having a first side and a second side; and
a recess formed between the first side and the second side, the recess having a shape corresponding to a median nerve on a palmar surface of a hand so that pressure on a median nerve is relieved.
